SEQUENCE LISTING (4 sequences)

SEQ ID NO: 1 - DNA, 2501 bp, Oryza sativa
  CDS (121)...(2295)
cggcacgagattttgcagtctcctctcctcctccgctcgagcgagtgagtcccgaccacgtcgctgccctcgcctcaccgccggccaaccgccgtgacgagagatcgagcagggcggggcatggacgagccttcgccgcgcggaggtgggtgcgccggggagccgccccgcatccggaggttggaggagtcggtggtgaaccgcatcgcggcgggggaggtgatccagcggccgtcgtcggcggtgaaggagctcatcgagaacagcctcgacgctggcgcctccagcgtctccgttgcggtgaaggacggtggcctcaagctcatccaggtctccgatgacggccatggcatcaggtttgaggatttggcaatattgtgcgaaaggcatactacctcaaagttatctgcatacgaggatctgcagaccataaaatcgatggggttcagaggggaggctttggctagtatgacttatgttggccatgttaccgtgacaacgataacagaaggccaattgcacggctacagggtttcttacagagatggtgtaatggagaatgagcctaagccttgcgctgcggtgaaaggaactcaagtcatggttgaaaatctattttacaacatggtagcccgcaagaaaacattgcagaactccaatgatgactaccccaagatcgtagacttcatcagtcggtttgcagtccatcacatcaacgttaccttctcttgcagaaagcatggagccaatagagcagatgttcatagtgcaagtacatcctcaaggttagatgctatcaggagtgtctatggggcttctgtcgttcgtgatctcatagaaataaaggtttcatatgaggatgctgcagattcaatcttcaagatggatggttacatctcaaatgcaaattatgtggcaaagaagattacaatgattcttttcataaatgataggcttgtagactgtactgctttgaaaagagctattgaatttgtgtactctgcaacattgcctcaagcatccaaacctttcatatacatgtccatacatcttccatcagaacacgtggatgttaatatacacccaaccaagaaagaggttagccttttgaatcaagagcgtattattgaaacaataagaaatgctattgaggaaaaactgatgaattctaatacaaccaggatattccaaactcaggcattaaacttatcagggattgctcaagctaacccacaaaaggataaggtttctgaggccagtatgggttctggaacaaaatctcaaaaaattcctgtgagccaaatggtcagaacagatccacgcaatccatctggaagattgcacacctactggcacgggcaatcttcaaatcttgaaaagaaatttgatcttgtatctgtaagaaatgttgtaagatcaaggagaaaccaaaaagatgctggtgatttgtcaagccgtcatgagctccttgtggaaatagattctagcttccatcctggccttttggacattgtcaagaactgcacatatgttggacttgccgatgaagcctttgctttgatacaacacaatacccgcttataccttgtaaatgtggtaaatattagtaaagaacttatgtaccagcaagctttgtgccgttttgggaacttcaatgctattcagctcagtgaaccagctccacttcaggagttgctggtgatggcactgaaagacgatgaattgatgagtgatgaaaaggatgatgagaaactggagattgcagaagtaaacactgagatactaaaagaaaatgctgagatgattaatgagtacttttctattcacattgatcaagatggcaaattgacaagacttcctgttgtactggaccagtacacccctgatatggaccgtcttccagaatttgtgttggctttaggaaatgatgttacttgggatgacgagaaagagtgcttcagaacagtagcttctgctgtaggaaacttctatgcacttcatcccccaatccttccaaatccatctgggaatggcattcatttatacaagaaaaatagagattcaatggctgatgaacatgctgagaatgatctaatatcagatgaaaatgacgttgatcaagaacttcttgcggaagcagaagcagcatgggcccaacgtgagtggaccattcagcatgtcttgtttccatccatgcgacttttcctcaagcccccgaagtcaatggcaacagatggaacgtttgtgcaggttgcttccttggagaaactctacaagatttttgaaaggtgttagctcataagtgagaaaatgaaggcagagtaagatcatgattcatggagtgtttttgaaaatgtgtataatttcaccgtattatgtactttgatagtgtctgtagaaactgaagaaagaaagatggctttacttctgaattgaaagttaacgatgccagcaattgtatattctgatcaaccaaaaaaaaaaaaaaaaaaaaaaaaaaa

SEQ ID NO: 2 - protein, 724 aa, Oryza sativa
MDEPSPRGGGCAGEPPRIRRLEESVVNRIAAGEVIQRPSSAVKELIENSLDAGASSVSVAVKDGGLKLIQVSDDGHGIRFEDLAILCERHTTSKLSAYEDLQTIKSMGFRGEALASMTYVGHVTVTTITEGQLHGYRVSYRDGVMENEPKPCAAVKGTQVMVENLFYNMVARKKTLQNSNDDYPKIVDFISRFAVHHINVTFSCRKHGANRADVHSASTSSRLDAIRSVYGASVVRDLIEIKVSYEDAADSIFKMDGYISNANYVAKKITMILFINDRLVDCTALKRAIEFVYSATLPQASKPFIYMSIHLPSEHVDVNIHPTKKEVSLLNQERIIETIRNAIEEKLMNSNTTRIFQTQALNLSGIAQANPQKDKVSEASMGSGTKSQKIPVSQMVRTDPRNPSGRLHTYWHGQSSNLEKKFDLVSVRNVVRSRRNQKDAGDLSSRHELLVEIDSSFHPGLLDIVKNCTYVGLADEAFALIQHNTRLYLVNVVNISKELMYQQALCRFGNFNAIQLSEPAPLQELLVMALKDDELMSDEKDDEKLEIAEVNTEILKENAEMINEYFSIHIDQDGKLTRLPVVLDQYTPDMDRLPEFVLALGNDVTWDDEKECFRTVASAVGNFYALHPPILPNPSGNGIHLYKKNRDSMADEHAENDLISDENDVDQELLAEAEAAWAQREWTIQHVLFPSMRLFLKPPKSMATDGTFVQVASLEKLYKIFERC

SEQ ID NO: 3 - DNA, 2381 bp, Arabidopsis thaliana
  CDS (3)...(2213)
agatgatcgacgattcgtctcttacggcggagatggaggaggaagaatctccggcgacgacgattgtaccgagagagccaccgaagattcaacgcttagaagaatcagtagtcaaccgtatcgcagctggtgaagtaatccagcgtccagtttcagctgtgaaagagctcgttgagaacagcctcgacgccgattcaagttccataagcgtcgttgtcaaagacggtggtttgaaactcattcaagtctccgacgacggtcacggtattagacgtgaagacttgccgatactatgcgagagacatacaacatcgaagctgactaagtttgaggatttgttctctctgagttcaatgggatttagaggagaggcattagctagtatgacctatgttgctcatgttacagtgactactattactaaaggccagattcatggttatagagtgtcttatagagatggtgtcatggagcatgaaccaaaggcgtgtgctgctgtcaaaggaacacagataatggtggagaatttgttctacaatatgattgctagaaggaagacacttcaaaattctgctgatgattacgggaaaatcgtggatttgctgagccggatggctattcattacaataatgtcagcttttcttgtcgaaagcatggagctgttaaggctgatgttcactcagtcgtgtcaccttcaaggcttgattcaattaggtctgtatatggagtatcagttgcaaagaacttgatgaaagtagaagtttcctcctgtgactcctctggttgtacttttgatatggagggtttcatatccaattctaactatgttgctaagaagactatattggtgcttttcattaatgatagattggtggaatgctctgccttaaaaagagccattgaaattgtttatgctgcaacattgccaaaagcatcaaaaccttttgtctacatgtcaatcaatttgccacgggaacatgttgatatcaatattcacccaacaaagaaagaggttagccttctaaaccaggaaatcattattgagatgatacagtcagaggttgaagtaaaactgagaaacgcaaatgatactaggacgtttcaagagcagaaagtggaatacattcaatctacgttaacatctcagaaaagtgattctccagtttctcagaagccttctggacaaaagacacagaaagttcctgtgaacaaaatggtgagaacagattcatcagatccagctggaaggttacatgcctttttgcaacccaagccacaaagtctccctgacaaggtttctagtttgagtgtagtaaggtcttctgtaaggcaaagaagaaacccaaaggaaactgctgatctttctagtgtccaggaacttattgctggagttgacagctgctgccatccaggtatgctggagactgtaaggaattgcacatatgttggaatggcagatgatgtttttgctttagttcagtataacacccatctatatctagcaaatgtggtgaatctcagcaaagagctaatgtatcagcaaactcttcgtcgttttgctcattttaacgcaatacagcttagcgatccagcccctttgtcagagttgatattgttggctctgaaagaggaggatctagatccaggaaatgatacaaaagatgatctgaaagaaagaattgctgaaatgaatacagaactcctcaaggaaaaagcagaaatgttagaggagtatttcagcgtgcacattgactccagtgcaaatttgtcaaggcttcctgtgatactcgaccagtatacacctgacatggatcgtgttcctgaatttttactatgcttgggaaatgatgttgagtgggaagatgagaagagttgctttcaaggagtttctgcagctattgggaacttttacgccatgcatcctcctcttttgccaaacccatcgggtgacggtattcagttctatagtaagagaggtgagagctctcaggaaaagtcagatttagagggtaacgtcgatatggaggacaatcttgaccaagatcttctgtcagatgctgaaaacgcatgggcacaacgtgaatggtcaatccaacacgtgttgtttccgtcaatgagattgttcttgaagccaccagcttccatggcttcaaatgggacttttgtaaaggtagcatcccttgaaaagctgtacaagatattcgaacgatgctaactgaaaccgctgattgtagaagaacttttgatatgagtagcttccatttgctctaactatgtttctagactttgaatgaaagtggaaccagtttacggttaaaccaaactgtggcacacacgactgaccaaaaccataacaatcaaactccaccttttcctgtga

SEQ ID NO: 4 - protein, 737 aa, Arabidopsis thaliana
MIDDSSLTAEMEEEESPATTIVPREPPKIQRLEESVVNRIAAGEVIQRPVSAVKELVENSLDADSSSISVVVKDGGLKLIQVSDDGHGIRREDLPILCERHTTSKLTKFEDLFSLSSMGFRGEALASMTYVAHVTVTTITKGQIHGYRVSYRDGVMEHEPKACAAVKGTQIMVENLFYNMIARRKTLQNSADDYGKIVDLLSRMAIHYNNVSFSCRKHGAVKADVHSVVSPSRLDSIRSVYGVSVAKNLMKVEVSSCDSSGCTFDMEGFISNSNYVAKKTILVLFINDRLVECSALKRAIEIVYAATLPKASKPFVYMSINLPREHVDINIHPTKKEVSLLNQEIIIEMIQSEVEVKLRNANDTRTFQEQKVEYIQSTLTSQKSDSPVSQKPSGQKTQKVPVNKMVRTDSSDPAGRLHAFLQPKPQSLPDKVSSLSVVRSSVRQRRNPKETADLSSVQELIAGVDSCCHPGMLETVRNCTYVGMADDVFALVQYNTHLYLANVVNLSKELMYQQTLRRFAHFNAIQLSDPAPLSELILLALKEEDLDPGNDTKDDLKERIAEMNTELLKEKAEMLEEYFSVHIDSSANLSRLPVILDQYTPDMDRVPEFLLCLGNDVEWEDEKSCFQGVSAAIGNFYAMHPPLLPNPSGDGIQFYSKRGESSQEKSDLEGNVDMEDNLDQDLLSDAENAWAQREWSIQHVLFPSMRLFLKPPASMASNGTFVKVASLEKLYKIFERC

(19) United States
(12) Patent Application Publication
(10) Pub. No.: US 2002/0088021 A1
(43) Pub. Date: Jul. 4, 2002

(54) Rice MLH1 ortholog and uses thereof

(76) Inventor: Pramod B. Mahajan, Urbandale, IA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/954,950

(22) Filed: Sep. 18, 2001

Related U.S. Application Data
(60) Provisional application No. 60/233,124, filed on Sep. 18, 2000.

(51) Int. Cl. A01H 5/00; C07H 21/04; C12N 9/22; C12N 5/04

(52) U.S. Cl. 800/278; 435/410; 435/199; 435/320.1; 536/23.2; 800/320.2

(57) ABSTRACT

Compositions and methods for inhibiting the cellular mismatch repair system in a plant host cell are provided. Compositions include the cDNA and amino acid sequence of a rice MLH1 ortholog. The nucleic acid molecules and proteins of the invention find use in increasing the efficiency of targeted gene mutation and homologous recombination in plants via inhibition of the plant cellular mismatch repair system. The plant cellular mismatch repair system is inhibited through the use of transposon tagging of a MLH1 gene, sense- and antisense-suppression of a MLH1 gene, antibody binding to a MLH1 polypeptide or variant polypeptide, targeted mutagenesis of specific amino acid residues of a plant MLH1 gene, and competition with a mismatch repair impaired MLH1 polypeptide through transgeneic over-expression of the impaired polypeptide. Also provided are transformed plant cells, plant tissues, plants, and seeds. Additional methods that are provided include the detection of as little as one base pair mismatch in a DNA duplex and the generation of plants with reversible male sterility for applications in hybrid generation.

### CROSS-REFERENCE TO RELATED APPLICATION

This application claims the benefit of U.S. Provisional Application Serial No. 60/233,124, filed Sep. 18, 2000, the content of which is herein incorporated by reference in its entirety.

### FIELD OF THE INVENTION

The invention relates to the genetic manipulation of plants, particularly to increasing the efficiency of targeted gene mutation and homologous recombination through inhibition of the cellular mismatch repair system.

### BACKGROUND OF THE INVENTION

Mismatched base pairing in DNA duplexes may arise due to errors introduced during DNA replication (Kornberg and Baker (1991) in DNA Replication (W. H. Freeman & Co., New York); Echols and Goodman (1991) Annu. Rev. Biochem. 60:477-511), heteroduplex formation during homologous recombination (Holliday (1964) Genet. Res 5:282-304; Petes and Hill (1988) Annu. Rev. Genet. 22:147-168) as a consequence of mutation, as well as by enzymatic modification of DNA such as deamination of 5-methylcytosine. These mismatches can lead to genome instability. Therefore, all living systems have evolved specialized pathways to repair specific mismatches that are somewhat different than other DNA repair mechanisms such as base excision repair and nucleotide excision repair (Wildenberg and Messelson (1975) Proc. Natl. Acad. Sci. USA 72:2202-2206; Wagner and Messelson (1996) Proc. Natl. Acad. Sci. USA 73:4136-4139; Radman and Wagner (1986) Annu. Rev. Genet. 20:523-538; Freidberg (1985) in DNA Repair (W. H. Freeman & Co., New York)).

Early studies in prokaryotic systems, especially Eschericia coli, led to the identification of one of these pathways, called the long-patch repair system or the methyl-directed mismatch repair system (Radman and Wagner (1986) Annu. Rev. Genet. 20:523-538). This pathway exhibits rather broad specificities for repairing mismatches generated during DNA biosynthesis as well as recombination. Several genes essential for the methyl-directed mismatch repair have been identified in E. coli. Primary among these are mutS, mutL, mutH, UvrD, and the Dam methyltransferase and exonuclease genes (Freidberg (1985) in DNA Repair (W. H. Freeman & Co., New York)).

Many of the mismatch repair genes and the pathways they participate in are also conserved in eukaryotic organisms (Nickoloff and Hoeskstra (1998) DNA Damage and Repair, Vol. I-II (Humana Press, New York); Muster-Nassal and Kolodner (1986) Proc. Natl. Acad. Sci. USA 83:7618-7622). Yeast PMS1 was one of the first eukaryotic mismatch repair genes to be isolated and shown to be an ortholog of bacterial mutL (Kramer et al. (1989) J. Bacteriol. 171:5359-5346). The genome of the yeast Saccharomyces cerevisiae has been completely sequenced and contains a total of four mutL homologs (Flores-Rozas and Kolodner (1998) Proc. Natl. Acad. Sci. 95:12404-12409. Orthologs of mutL have also been isolated from mouse (Edelmann et al. (1996) Cell 85:1125-1134), human (Bronner et al. (1994) Nature 368:258-261), and rat (Geeta et al. (1999) Genomics 62:460-467). In humans, three mutL homologs have been cloned (MLH1, PMS1, and PMS2) (Bronner et al. (1994) Nature 368:258-261; Nicolaides et al. (1994) Nature 371:75-80; Papadopoulos et al. (1994) Science 263:1625-1629).

Less is known about the mismatch repair system in plants. Four Arabidopsis thaliana mutS homologs have been reported (AtMSH2, AtMSH3, AtMSH6-1, and AtMSH6-2) (Culigan and Hays (1997) Plant Physiol. 115:833-839; Ade et al. (1999) Mol. Gen. Genet. 262:239-249) and, as has generally been the case in other eukaryotes, this suggests that plants similarly possess gene families whereas prokaryotes rely on a single gene. Recently, Jean et al. reported the cloning and characterization of the first plant mutL ortholog from Arabidopsis thaliana (AtMLH1) (Jean et al. (1999) Mol. Gen. Genet 262:633-342).

The sequence conservation of the mutL orthologs of bacteria, yeast, and mammals has facilitated the characterization of the principle players involved in this important mismatch repair pathway. Furthermore, the phenotypes of mismatch repair deficient mutants are also similar and have indicated the involvement of the proteins encoded by the mutL orthologs in DNA damage surveillance, transcription-coupled repair, and recombinogenic and meiotic processes. Mismatch repair has the critical role of stabilizing the cellular genome by correcting DNA replication errors and by blocking recombination events between divergent DNA sequences.

Evidence for the role of some eukaryotic mismatch repair proteins in meiotic processes can be found in experiments with knockout mice. For example, mice that are homozygous for a null mutation in the MSH2 gene breed normally (de Wind et al. (1995) Cell 82:321-330; Reitmair et al. (1995) Genet. 11:64-70). Interestingly, in mice with PMS2 mutations, the males are sterile and the females normal (Baker et al. (1995) Cell 82:309-320). On the other hand, in homozygous mice with MLH1 null mutations, both the males and females are unable to reproduce (Edelmann et al. (1996) Cell 85:1125-1134). Furthermore, inactivation of hHR6B, the human ortholog of the yeast ubiquitin-conjugating enzyme RAD6, causes male infertility through the derailment of spermatogenesis during the postmeiotic condensation of chromatin in spermatids. Heterozygous male mice and homozygous female mice appear completely normal and are fertile and thus able to transmit the defect (Roest et al. (1996) Cell 86:799-810).

The mismatch repair proteins have important roles in mismatch repair, recombination, and stabilization of the cellular genome and, thus, have applications in transgenic systems. The only plant mutL ortholog cloned to date is from A. thaliana. Thus, other plant mismatch repair sequences are needed.

### SUMMARY OF THE INVENTION

The present invention discloses a rice ortholog of mutL. Sequence comparisons indicate that this cDNA belongs to the MLH1 class of mutL orthologs and has accordingly been named rice MLH1. This rice cDNA has a variety of applications including altering the efficiency of targeted gene mutation and homologous recombination through modulation of the plant cellular mismatch repair system, the induction of male sterility in monocots for applications in hybrid generation, and use as a reagent in mismatch detection, in in vitro mismatch repair, and in in vitro mismatch repair assays.

Compositions and methods for inhibiting the cellular mismatch repair system in a plant host cell are provided. Particularly, the complete cDNA and amino acid sequence of a rice MLH1 ortholog are provided. The nucleic acid molecules and proteins of the invention find use in increasing the efficiency of targeted gene mutation and homologous recombination. This increase in mutagenesis efficiency facilitates the genetic modification of plants for applications including, but not limited to, agronomics, insect resistance, disease resistance, herbicide resistance, sterility, grain characteristics, and commercial products. Furthermore, an increased efficiency of homologous recombination enables the generation of hybrid plant species that would not be possible to obtain using conventional breeding techniques.

The methods of the invention are directed to the inhibition of the plant cellular mismatch repair system to increase the efficiency of targeted gene mutation and homologous recombination. The plant cellular mismatch repair system is inhibited through the use of transposon tagging of an MLH1 gene, sense- and antisense-suppression of an MLH1 gene, antibody binding to an MLH1 polypeptide or variant polypeptide, targeted mutagenesis of specific amino acid residues encoded by an MLH1 gene, and competition with a mismatch repair impaired MLH1 polypeptide through transgenic over-expression of the impaired polypeptide. In particular, methods are provided for the transient inhibition of the plant cellular mismatch repair system. Also provided are transformed plant cells, plant tissues, plants, and seeds. Additional methods that are provided include the detection of single or multiple base pair mismatches in a DNA duplex, and the generation of plants with male sterility for applications in hybrid generation.

### BRIEF DESCRIPTION OF THE DRAWINGS

FIG. 1 provides the nucleotide sequence (SEQ ID NO:1) of the rice MLH1 cDNA and the amino acid sequence (SEQ ID NO:2) for the encoded rice MLH1 protein.

FIG. 2 displays the amino acid sequence of the rice MLH1 protein (SEQ ID NO:2). The region of homology with the yeast mutL signature sequence is highlighted in bold.

FIG. 3 shows an alignment of the rice MLH1 amino acid sequence (SEQ ID NO:2) (top strand) with that of the Arabidopsis thaliana MLH1 (SEQ ID NO:4). These proteins display 74.4% similarity and 66.6% identity.

FIG. 4 shows an alignment of the nucleotide sequence of rice MLH1 cDNA (SEQ ID NO:1) (top strand) with that of the A. thaliana MLH1 (Accession No. AJ012747; SEQ ID NO:3). Overall, these sequences are 67.9% identical as determined by the BESTFIT program of GCG. Parameters used with BESTFIT were as follows: Gap Weight: 50; Ave. Match: 10.0; Length Weight: 3; Ave. Mismatch: −9.0; Quality: 7470; Length: 2188; Ratio: 3.484; Gaps: 10.

### DETAILED DESCRIPTION OF THE INVENTION

Nucleotide sequences and proteins useful for increasing the efficiency of targeted gene mutation and homologous recombination are provided. The nucleotide and amino acid sequences correspond to a rice MLH1 cDNA. MLH1 is an ortholog of the E. coli. mutL gene. Orthologs of mutL have been isolated from a number of species including yeast, mouse, human, rat, and Arabidopsis. The mutL gene encodes an enzyme that is part of the methyl directed mismatch repair system with broad specificity for repairing mismatches generated during DNA biosynthesis and recombination. The MLH1 sequences of the invention find use in increasing the efficiency of targeted gene mutation and homologous recombination through the inhibition of the DNA mismatch repair system.

Compositions of the invention include MLH1 nucleotide and amino acid sequences that are involved in modulating DNA repair and recombination. In particular, the present invention provides for an isolated nucleic acid molecule comprising nucleotide sequences encoding the amino acid sequence shown in SEQ ID NO:2. The present invention also provides the nucleotide sequence encoding the DNA sequence deposited in a bacterial host as Patent Deposit No. PTA-2021. Further provided are polypeptides having an amino acid sequence encoded by a nucleic acid molecule described herein, for example that set forth in SEQ ID NO:1, that has been deposited in a bacterial host as Patent Deposit No. PTA-2021, and fragments and variants thereof.

Plasmids containing the nucleotide sequence of the invention were deposited with the Patent Depository of the American Type Culture Collection (ATCC), Manassas, Va., on Jun. 13, 2000 and assigned Patent Deposit No. PTA-2021. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. §112.

The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequence and protein encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence function in the mismatch repair system. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to 2283 nucleotides or the full-length nucleotide sequence encoding the protein of the invention.

A fragment of the rice MLH1 cDNA (SEQ ID NO:1) that encodes a biologically active portion of the MLH1 protein of the invention will encode at least 20, 25, 30, 50, 75, 100, 150, 200, 250, 300, 350, 400, 450, 500, 550, 600, 650, 700, or 724 contiguous amino acids, or up to the total number of amino acids present in the full-length MLH1 protein of the invention (for example, 724 amino acids for SEQ ID NO:2). Fragments of SEQ ID NO:1 that are useful as hybridization probes or PCR primers generally need not encode a biologically active portion of an MLH1 protein.

Thus, a fragment of SEQ ID NO:1 may encode a biologically active portion of an MLH1 protein, or it may be a fragment that can be used as a hybridization probe or PCR primer using methods disclosed below. A biologically active portion of the MLH1 protein can be prepared by isolating a portion of the disclosed nucleotide sequence expressing the encoded portion of the MLH1 protein (e.g., by recombinant expression in vitro), and assessing the activity of the encoded portion of the MLH1 protein. Nucleic acid molecules that are fragments of MLH1 comprise at least 27, 28, 29, 30, 40, 50, 60, 75, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900,2000,2100,2200, or up to 2283 nucleotides of SEQ ID NO:1.

By “variants” is intended substantially similar sequences. For nucleotide sequences, conservative variants include those sequences that, because of the degeneracy of the genetic code, encode the amino acid sequence of the MLH1 polypeptide of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode an MLH1 protein of the invention. Generally, variants of a particular nucleotide sequence of the invention will have at least about 75%, 80%, 85%, 90%, 91%, 92%, 93%, 94%, 95%, 96%, 97%, 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, mismatch repair activity. Such variants may result from, for example, genetic polymorphism or from human manipulation. Biologically active variants of a native MLH1 protein of the invention will have at least about 75%, 76%, 77%, 78%, 79%, 80%, 85%, 90%, 91%, 92%, 93%, 94%, 95%, 96%, 97%, 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the MLH1 protein can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York), and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable when alteration of the biological activity of the protein is undesirable. In other cases, alteration of the endogenous biological activity of the protein may be desirable and non-conservative substitutions may be preferable.

Thus, the genes and nucleotide sequences of the invention include both the naturally occurring sequences as well as mutant forms. Likewise, the proteins of the invention encompass both naturally occurring proteins as well as variations and modified forms thereof. Obviously, the mutations that will be made in the DNA encoding the variant must not place the sequence out of reading frame and preferably will not create complementary regions that could produce secondary mRNA structure. See, EP Patent Application Publication No. 75,444.

When it is difficult to predict the exact effect of the substitution, deletion, or insertion in advance of doing so, one skilled in the art will appreciate that the effect will be evaluated by routine screening assays. That is, the activity can be evaluated by mismatch repair assays, described elsewhere herein. See, for example, Spampinato et al. (2000) J. Biol. Chem. 275:9863-9869, herein incorporated by reference.

Variant nucleotide sequences and proteins also encompass sequences and proteins derived from a mutagenic and recombinogenic procedure such as DNA shuffling. With such a procedure, one or more different MLH1 coding sequences can be manipulated to create a new MLH1 polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the MLH1 gene of the invention and other known genes to obtain a new gene coding for a protein with an improved property of interest. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequence of the invention can be used to isolate corresponding sequences from other plants. In this manner, methods such as PCR, hybridization, and the like can be used to identify such sequences based on their sequence homology to the sequence set forth herein. Sequences isolated based on their sequence identity to the entire MLH1 sequence set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences that encode for an MLH1 protein and which hybridize under stringent conditions to the MLH1 sequence disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²p, or any other detectable marker. Thus, for example, probes for hybridization can be made by labeling synthetic oligonucleotides based on the MLH1 sequence of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire MLH1 cDNA sequence disclosed herein, or one or more portions thereof, may be used as a probe capable of specifically hybridizing to corresponding MLH1 sequences and messenger RNAs. To achieve specific hybridization under a variety of conditions, such probes include sequences that are unique among MLH1 protein sequences and are at least about 10, 20, 27, 30, 40, 50, 60, or more than 60 nucleotides in length. Such probes may be used to amplify corresponding MLH1 sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1×to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5×to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (%GC)−0.61 (% form)−500/L; where M is the molarity of monovalent cations, %GC is the percentage of guanosine and cytidine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode for a MLH1 protein and which hybridize under stringent conditions to the MLH1 sequence disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 75%, 80%, 85%, 90%, 91%, 92%, 93%, 94%, 95%, 96%, 97%, 98%, or 99% sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, 65%, 70%, 75%, 80%, 85%, 90%, 91%, 92%, 93%, 94%, 95%, 96%, 97%, 98% or 99%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least about 70%, 75%, 80%, 85%, 90%, 91%, 92%, 93%, 94%, 95%, 96%, 97%, 98% or 99% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

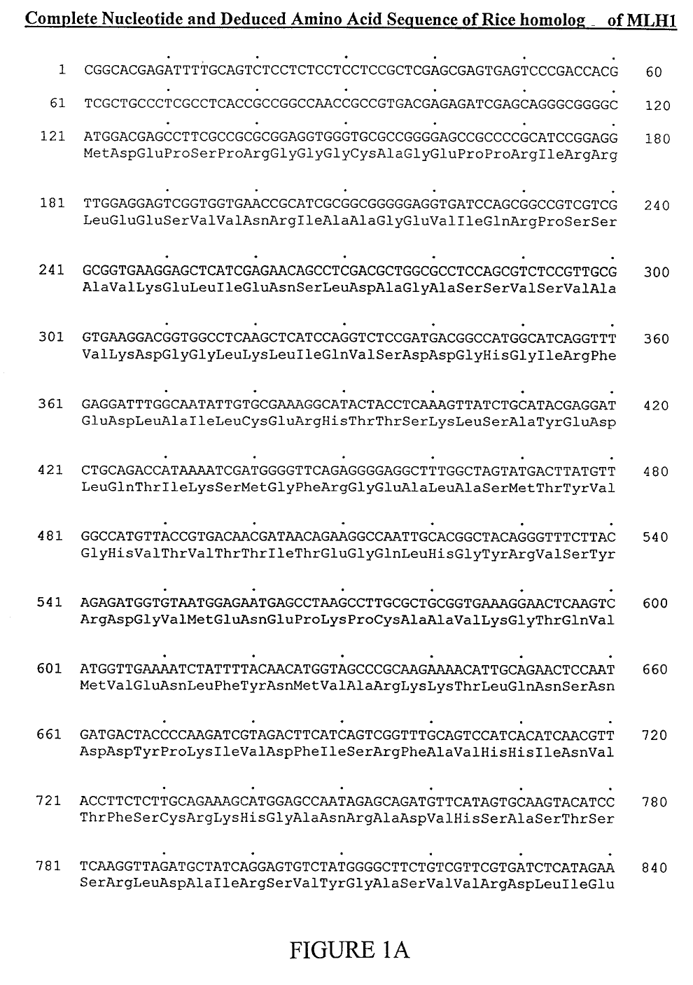

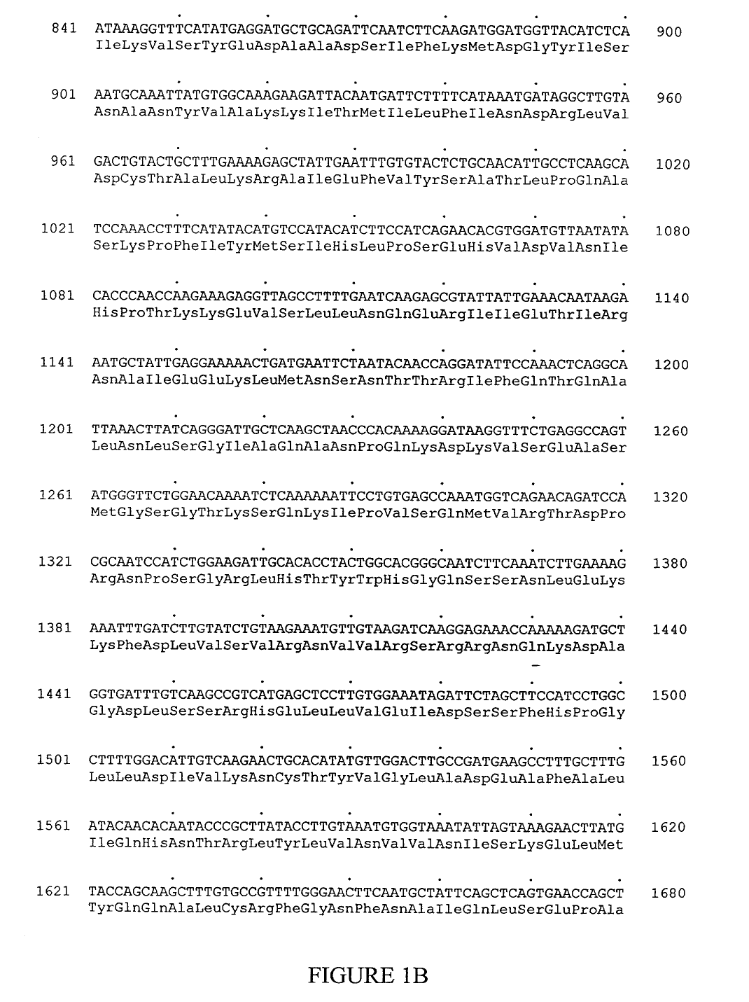

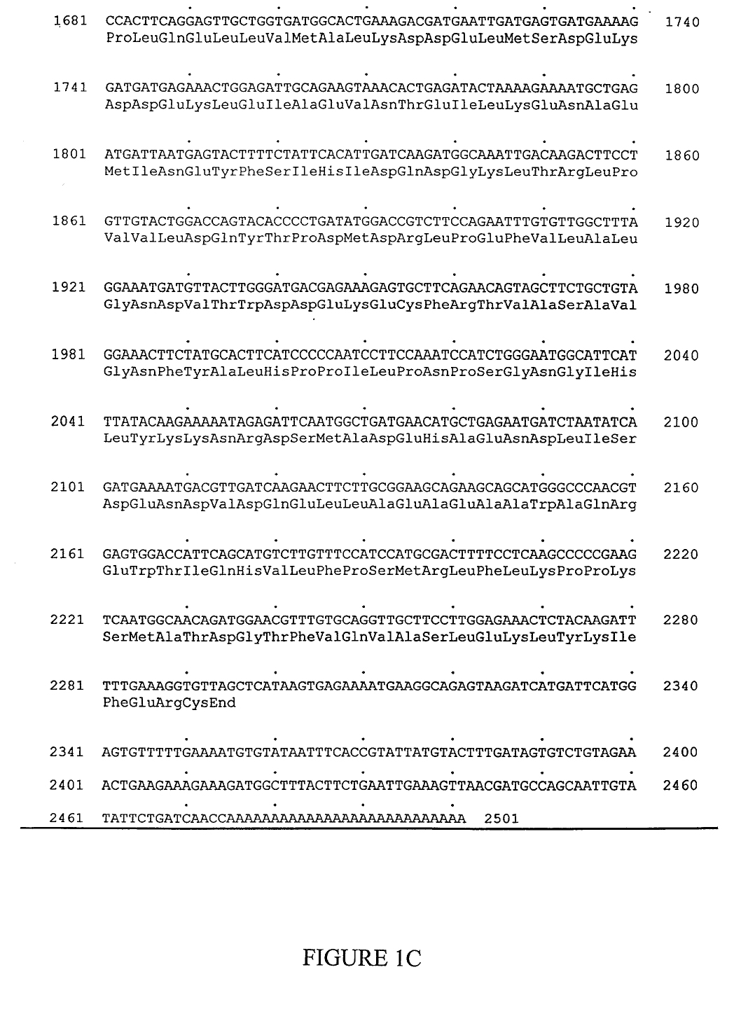

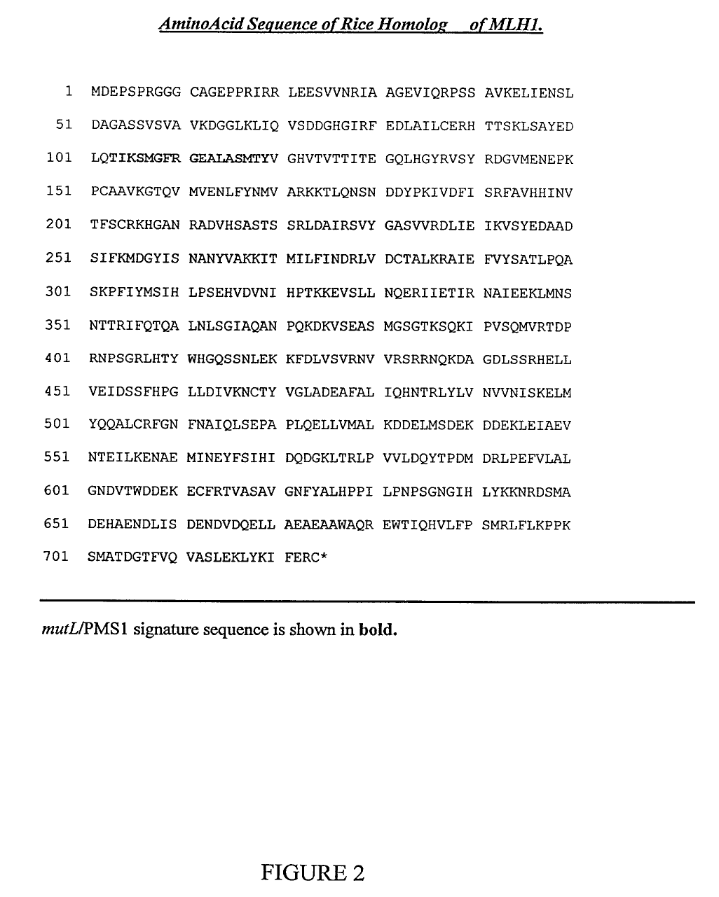

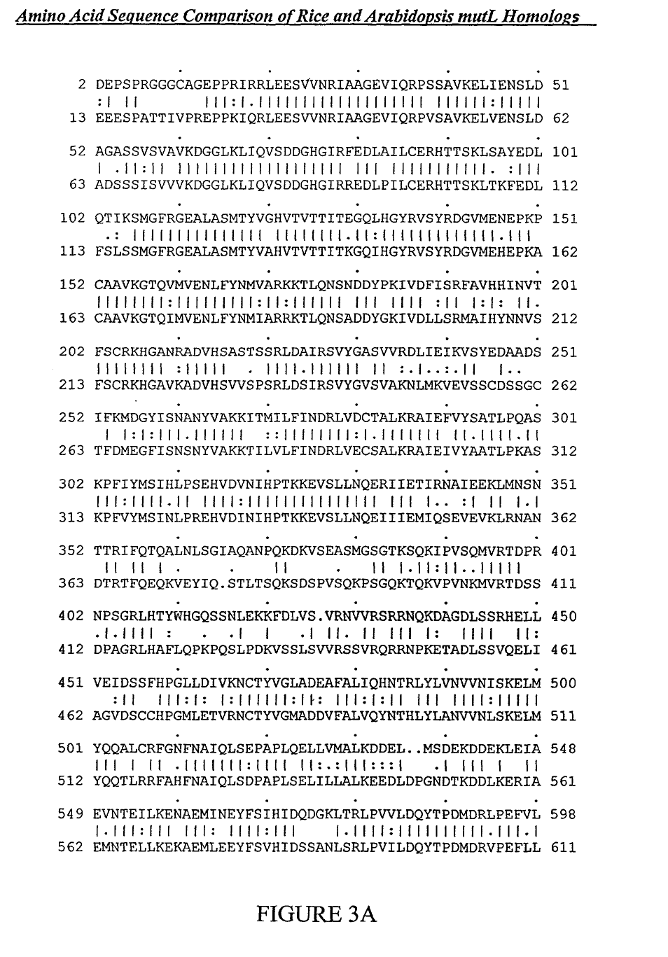

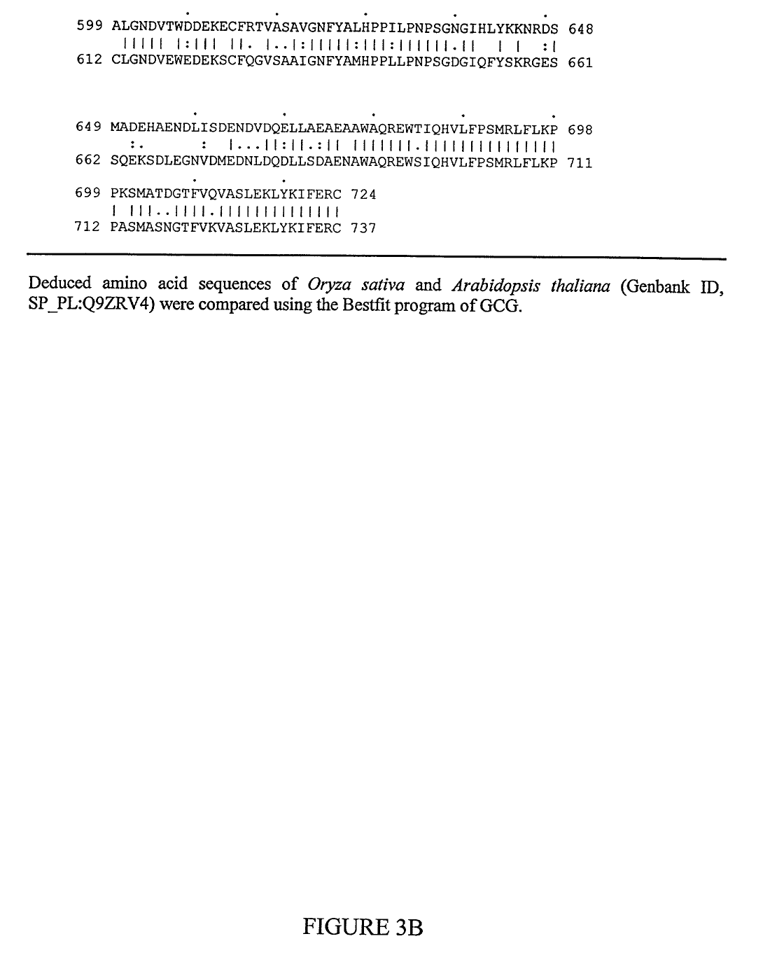

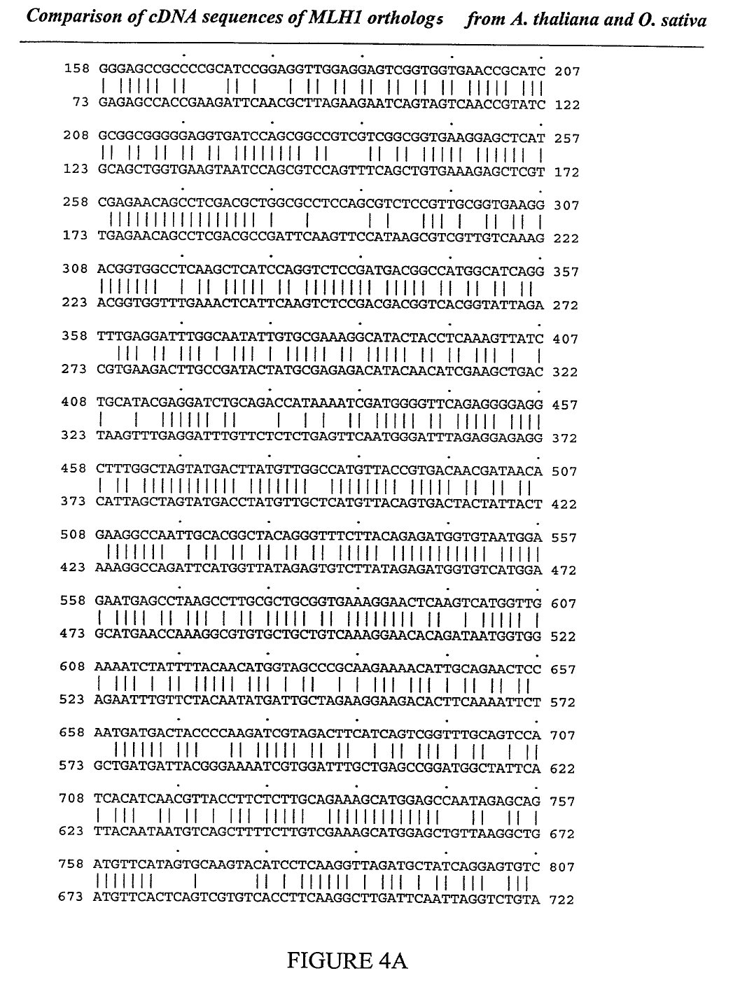

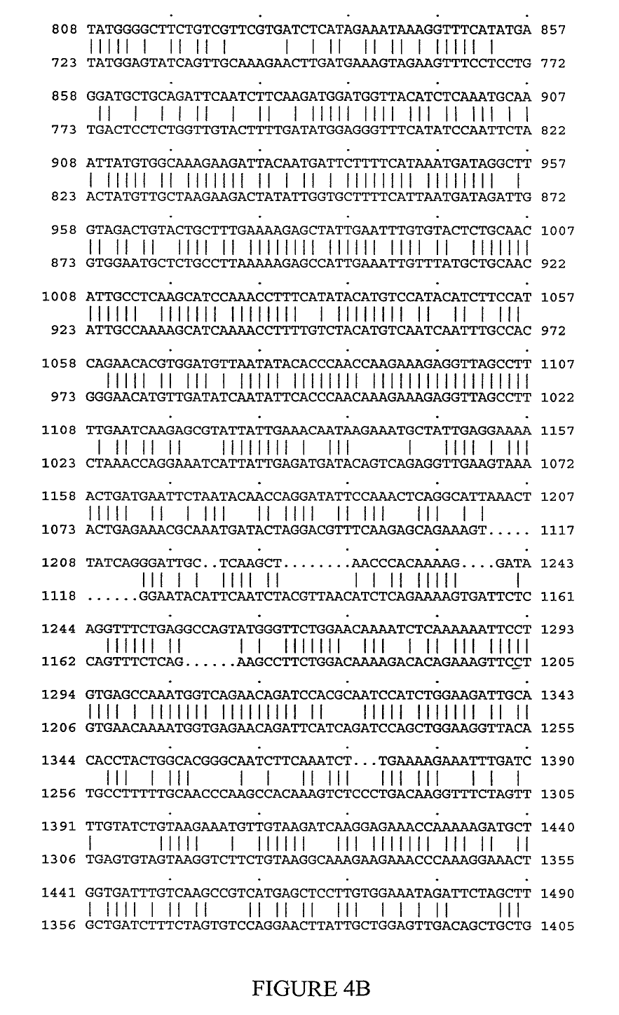

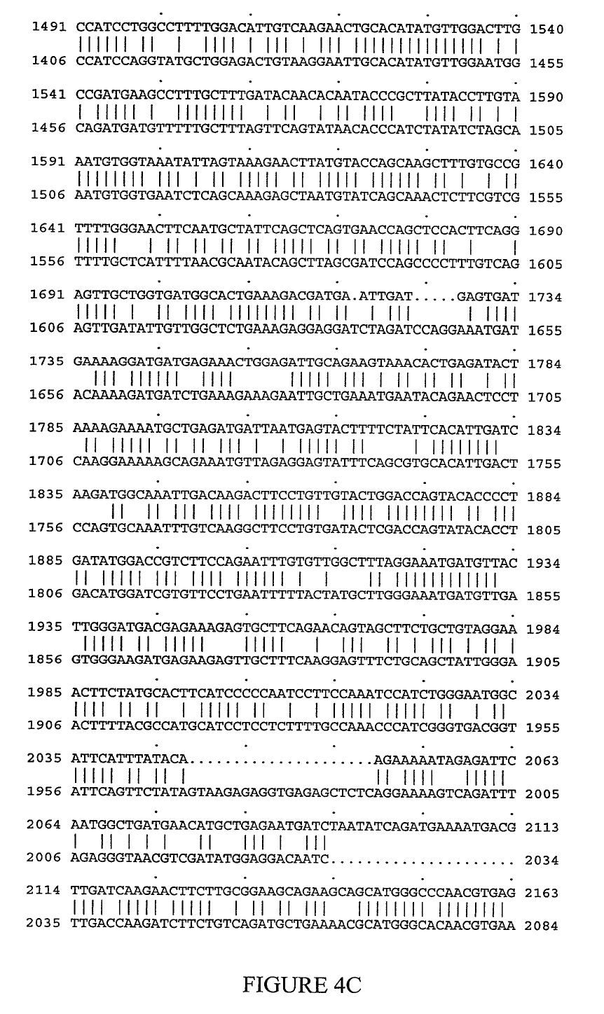

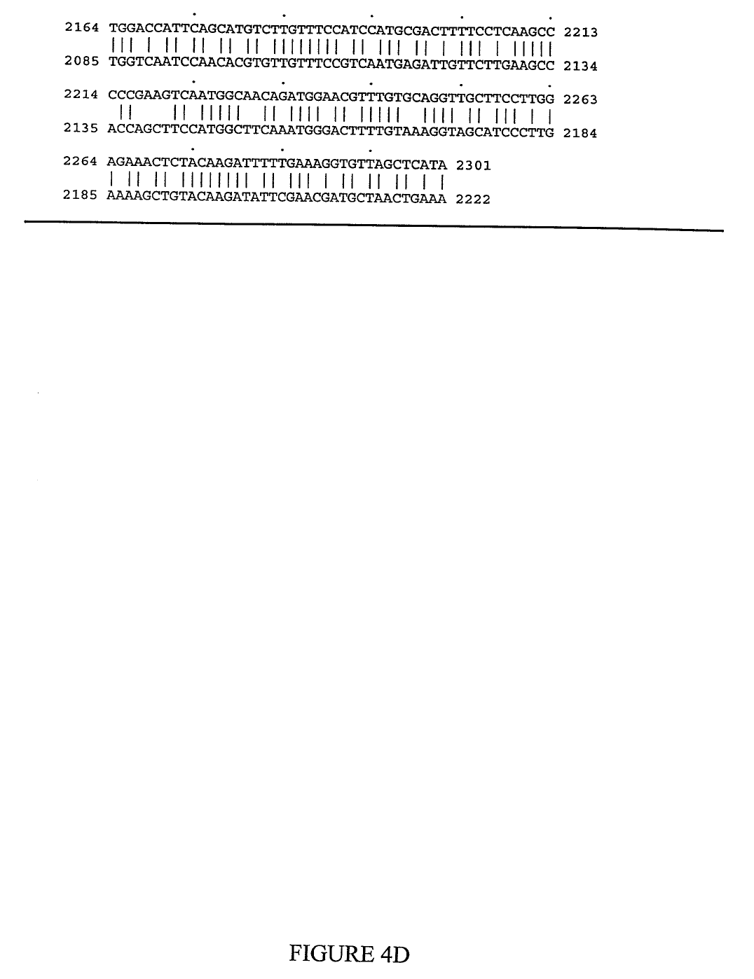

The rice MLH1 cDNA disclosed in the present invention (SEQ ID NO:1) encodes a 724 amino acid protein (FIG. 1; SEQ ID NO:2) and displays sequence similarity to the E. coli mutL orthologs from a variety of organisms including human, rat, mouse, fruit fly, C. elegans, and Arabidopsis. These mutL orthologs are part of the methyl-directed mismatch repair pathway that functions in the repair of mismatches generated during DNA biosynthesis and recombination. FIG. 2 displays the amino acid sequence of the rice MLH1 protein with the mutL signature sequence highlighted in bold. The first full-length mutL ortholog from a plant species that has been disclosed is that from Arabidopsis. FIG. 3 shows an alignment of the rice MLH1 amino acid sequence of the instant invention (SEQ ID NO:2) (top strand) with the MLH1 sequence of Arabidopsis (SEQ ID NO:4). These proteins display substantial sequence similarity and identity (74.4% and 66.6%, respectively). FIG. 4 shows an alignment of the nucleotide sequence of the rice MLH1 cDNA sequence of the present invention (SEQ ID NO:1) (top strand) with that of the A. thaliana MLH1 (SEQ ID NO:3). Again these sequences display substantial homology as they have an overall sequence identity of 67.9% as determined by the program BESTFIT.

Although the methyl-directed pathway for repair of DNA biosynthetic errors within the DNA helix has been demonstrated in a wide variety of species, the mechanisms and functions of mismatch correction are best understood in E. coli (see Modrich (1989) J. Biol. Chem. 264:6597-6600; Modrich (1991) Annu. Rev. Genet. 25:229-253; Modrich (1994) Science 266:1959-1960; Modrich et al. (1996) Annu. Rev. Biochem. 65:101-133; herein incorporated by reference.). The fidelity of DNA replication in E. coli is enhanced 100-1000 fold by this post-replication mismatch correction system. This system processes base pairing errors within the helix in a strand-specific manner by exploiting patterns of DNA methylation. Since DNA methylation is a post-synthetic modification, newly synthesized strands temporarily exist in an unmethylated state, with the transient absence of adenine methylation on GATC sequences directing mismatch correction to new DNA strands within the hemimethylated duplexes.

The mismatch correction system is capable in vivo of correcting differences between duplexed strands involving a single base insertion or deletion. Genetic analyses have demonstrated that the mismatch correction process requires intact genes for several proteins, including, but not limited to, the products of the mutH, mutL, and mutS genes, as well as DNA helicase II and single-stranded DNA binding protein (SSB). Specific components of the E. coli mispair correction system have been isolated and the biochemical functions determined (Lahue et al. (1989) Science 245:160). The MutS protein binds to each of the eight base pair mismatches and does so with differential efficiency (Su et al. (1988) J. Biol. Chem. 263:6829). Grilley et al. (1989) J. Biol. Chem. 264: 1000 demonstrate that the MutL protein interacts with the MutS protein heteroduplex DNA complex. MutL interacts with MutH and Helicase II (Mechanic et al. (2000) J. Biol. Chem. 275:38337-38346; Hall et al. (1999) J. Biol. Chem. 274:1306-1312; Yamaguchi et al. (1998) J. Biol. Chem. 273:9197-9201; herein incorporated by reference). The MutH protein is responsible for d(GATC) site recognition and possesses a latent endonuclease that incises the unmethylated strand of hemimethylated DNA 5′ to the G of d(GATC) sequences (Welsh et al.(1987) J. Biol. Chem. 262:15624).

Furthermore, a role for the E. coli mismatch repair system in controlling recombination between related but non-allelic sequences has also been indicated (Feinstein and Low (1986) Genetics 113:13; Rayssiguier (1989) Nature 342:396; Shen (1989) Mol. Gen. Genetics 218:358; Petit (1991) Genetics 129:327; Worth et al. (1994) Proc. Natl. Acad. Sci. 91:3238-3241; herein incorporated by reference). Normally the frequency of crossovers between sequences that differ by a few percent or more at the base pair level are rare, whereas in bacterial mutants deficient in methyl-directed mismatch repair, the frequency of such events increases dramatically. The largest increases are observed in mutS and mutL deficient strains (Rayssiguier, supra; and Petit, supra). In addition, the mutL orthologs and other proteins involved in DNA repair mechanisms play a role in the regulation of meiotic processes.

The present invention takes advantage of the important roles of the MLH1 proteins in mismatch repair, recombination, and meiotic processes. One aspect of the present invention is directed to the inhibition of either the expression or the activity of MLH1 proteins in plants, to impair the cellular mismatch repair system and consequently encourage genetic modifications through increased rates of mutagenesis and non-specific recombination events. For example, the methods of the present invention that are directed to the inhibition of the plant cellular mismatch repair system have use in increasing the efficiency of the method of genetic modification known as chimeraplasty (See, U.S. Pat. Nos. 5,565,350; 5,731,181; 5,756,325; 5,760,012; 5,795,972; and 5,871,984; all of which are herein incorporated by reference) and described herein infra. In this manner, it is also an object of the invention to facilitate the formation of novel hybrid species, or more specifically, novel hybrid genes or enzymes by in vivo intergeneric and/or interspecific recombinations. Sense and antisense oligonucleotides, antibodies, peptides, transposons, site-directed mutagenesis, ribozymes, and the like may be utilized to inhibit the mismatch repair activity of MLH1 proteins. Because mismatch repair mutants may be genetically unstable, it may be advantageous to use a transient inhibition of the mismatch repair system for only as long as necessary to construct the desired genetic modification, and then restore the system to normal. The present invention provides methods for such a transient inhibition of the cellular mismatch system.

Another aspect of the present invention is directed to the generation of plants with reversible male-sterility. The reversible male-sterile trait is enabled by transforming a plant with a genetic construct that includes regulatory elements and nucleotide sequences capable of acting in a fashion to inhibit pollen formation or function, thus rendering the transformed plant reversibly male-sterile. In particular, the present invention involves inhibiting the expression of an MLH1 gene of the invention with the methods of co-suppression or antisense suppression through the use of an anther-specific promoter. Male sterility is reversed by incorporation into a plant of a second nucleic acid construct that represses the expression of the inhibiting nucleic acid molecules.

It is also an object of the present invention to provide methods for using the rice MLH1 protein of the invention, and variants or fragments thereof, alone or in combination with other proteins, for detecting and localizing base pair mismatches in double-stranded nucleic acid molecules including, but not limited to, duplex DNA molecules, particularly those double-stranded nucleic acid molecules comprising several kilo base pairs. The detection of mutations and identification of similarities or differences in DNA has important applications in increasing the world food supply by developing disease resistant and/or higher yielding crop strains, in forensic science, in the study of evolution and populations, and in scientific research in general (Guyer et al. (1995) Proc. Natl. Acad. Sci. USA 92:10841; Cotton (1997) TIG 13:43). One particular application is the identification of single nucleotide polymorphisms (SNP's). The further manipulation of nucleic acid molecules containing mismatches is also an object of the invention as will become apparent from the description of the relevant embodiments.

The various embodiments of the present invention that are directed to the inhibition of the plant cellular mismatch repair system are described below.

In the following embodiments of the invention a plant nucleotide sequence encoding an MLH1 protein is mutated, to decrease or obliterate the activity of the encoded MLH1 protein, and thus impair the cellular mismatch repair system. By “mismatch repair system” is intended the primary mechanism for repair of replication errors in E. coli and the homologous mismatch repair systems that have been identified in eukaryotic systems ranging from yeast to humans. The sequence of biochemical reactions that comprise the mismatch repair pathway have been most described in E. coli, and the proteins responsible for each step are known. For reviews, see Modrich (1989) J. Biol. Chem. 264:6597-6600; Modrich (1991) Annu. Rev. Genet. 25:229-253; Modrich (1994) Science 266:1959-1960; Modrich et al. (1996) Annu. Rev. Biochem. 65:101-133; herein incorporated by reference. By “mismatch repair activity” is intended the enzymatic activity of the polypeptide that is involved in the mismatch repair system. Methods for assaying mismatch repair activity are known to one of skill in the art and include, but are not limited to, in vitro mismatch repair assays, in vitro mismatch excision assays, nitrocellulose filter binding assays, gel mobility shift assays, helicase assays, d(GATC) specific endonuclease activity assays, and in vivo mutator assays.

By “mutated” is meant that one or more amino acids are altered relative to the native protein. A “defective cellular mismatch repair system” or an MLH1 protein with “defective mismatch repair activity” is one with an altered mismatch repair activity. The altered mismatch repair activity can be any change relative to that of the wild-type sequence including, but not limited to, reduced (relative to the wild-type or unmodified plant), obliterated mismatch repair activity, or increased mismatch repair activity. The genetic modification of a plant with such a defective cellular mismatch repair system is then facilitated, due to the increase in efficiency of targeted gene mutation and homologous recombination. Thus, transformation with nucleic acid containing desired mutation(s) or sequences to be homologously recombined in such a plant results in a higher number of transformants with the desired genetic modification.

In one example of this embodiment the method comprising transposon tagging is used to mutate the plant gene encoding the MLH1 protein. This method comprises insertion of a transposon within a plant MLH1 gene sequence to alter MLH1 gene expression and to, thus, alter the mismatch repair activity of the encoded MLH1 protein. As a result, the mismatch repair activity of the plant is similarly altered. Plants possessing such a mutated MLH1 gene are then transformed with nucleic acid containing the desired mutation(s) or sequences to be homologously recombined.

An embodiment of the invention is a plant cell grown in tissue culture wherein the mutated polypeptide produced by a nucleotide sequence of the invention alters the mismatch repair activity of the plant cell. The plant cell may be a member of a population of plant cells. The plant cells maybe cultured in vitro. The cultured plant cells with altered mismatch repair activity may be used for transformation with a nucleotide sequence of interest.

By “MLH1 gene” is meant a MutL homolog such as the MLH1 cDNA sequence set forth in SEQ ID NO:1. In this embodiment, a decrease in expression of the MLH1 protein of the invention is the goal, and insertion of a transposon within a regulatory region of this gene, in addition to, or rather than, an insertion within the MLH1 coding sequence, may result in decreased expression of the MLH1 protein. For this reason, a transposon that is within an exon, intron, 5′ or 3′ untranslated sequence, a promoter, or any other regulatory sequence of the MLH1 gene corresponding to the MLH1 cDNA of the invention, that results in decreased expression of the MLH1 protein, is also an object of this embodiment. Methods for the transposon tagging of specific genes in plants are well known in the art (see for example, Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928). In addition, the TUSC process for selecting Mu-insertions in selected genes has also been described (Benson et al. (1995) Plant Cell 7:75-84; Mena et al. (1996) Science 274:1537-1540; U.S. patent application Ser. No. 08/835,638, which is a continuation of U.S. patent application Ser. No. 08/262,056, both applications of which are herein incorporated by reference).

Plant transformants containing such a genetic modification as described above that results in decreased or obliterated expression of the MLH 1 protein of the invention are then selected by various methods known in the art. These methods include, but are not limited to, methods such as immunoblotting using antibodies that bind to the MLH1 proteins of interest, single nucleotide polymorphism (SNP) analysis, or assaying for the products of a reporter or marker gene, and the like.

In another example of this embodiment of the invention the activity of the plant MLH1 protein is altered through site-specific mutation of the genomic nucleotide sequence encoding the MLH1 protein. The mutagenesis is performed using methods known in the art such as, for example, chimeraplasty, described herein, infra. MLH1 polypeptides with altered mismatch repair activity are referred to herein as having “defective mismatch repair activity”. This method involves mutation of the codons corresponding to specific amino acids that are important or crucial for MLH1 enzyme activity such as, but not limited to, the amino acids that are conserved among the members of the MutL family. Suitable mutations include, but are not limited to, those described in Hall et al. (1999) J. Biol. Chem. 274:1306-1312; Spampinato et al. (2000) J. Biol. Chem. 275:9863-9869; and Guerrette et al. (1999) J. Biol. Chem. 274:6336-6341, herein incorporated by reference. Plants with an MLH1 gene encoding a polypeptide that is defective in mismatch repair activity are then transformed and selected for as described above.

In another example of this embodiment of the invention, transgenic co-suppression is used to alter the expression of the plant MLH1 gene. By “co-suppression” is intended the use of nucleotide sequences in the sense orientation to suppress the expression of the corresponding endogenous genes in plants. In the same manner as described in the previous embodiment, the genetic modification of a plant with such an inhibited cellular mismatch repair system is then facilitated upon transformation with DNA containing the desired mutation(s) or sequences to be homologously recombined, due to an increase in efficiency of targeted gene mutation and homologous recombination.

The method of co-suppression comprises transforming a plant cell with at least one expression cassette comprising a promoter that drives expression in the plant operably linked to at least one nucleotide sequence encoding an MLH1 protein. In this method, the inhibition of the cellular mismatch repair system is made transient, through the use of a chemical-inducible promoter in the above described expression cassette to drive expression of the MLH1 nucleotide sequence, such that inhibition of the cellular mismatch repair system only occurs when the chemical inducer is present. In this manner, a plant comprising such an expression cassette is transformed with nucleic acid containing the desired mutation(s) or sequences to be homologously recombined in the presence of a chemical compound capable of inducing the promoter of the expression cassette and, thus, inhibition of the cellular mismatch repair system. The chemical inducer is only present during the transformation procedure.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, greater than about 75%, 80%, 85%, 90%, or 95% sequence identity. See, U.S. Pat. Nos. 5,283,184 and 5,034,323; herein incorporated by reference.

The endogenous gene targeted for co-suppression may be a gene encoding any plant MLH1. For example, where the endogenous gene targeted for co-suppression is the rice MLH1 gene disclosed herein (SEQ ID NO:1), co-suppression is achieved using an expression cassette comprising the rice MLH1 gene sequence, or variant or fragment thereof.

In a related example of this embodiment, antisense suppression is used to reduce the level of MLH1 protein in a plant and inhibit the plant cellular mismatch repair system. By “antisense suppression” is intended the use of nucleotide sequences that are antisense to nucleotide sequence transcripts of endogenous plant genes to suppress the expression of those genes in the plant. This method comprises transforming a plant cell with at least one expression cassette comprising a promoter that drives expression in the plant cell operably linked to at least one nucleotide sequence that is antisense to a nucleotide sequence transcript of an MLH1 gene. In the same manner as described for the previous example, the inhibition of the cellular mismatch repair system is made transient, through the use of a chemical-inducible promoter to drive expression of the MLH1 antisense nucleotide sequence.

Methods for suppressing gene expression in plants using nucleotide sequences in the antisense orientation are known in the art. It is recognized that with these nucleotide sequences, antisense constructions, complementary to at least a portion of the messenger RNA (mRNA) for the MLH1 sequences can be constructed. The methods generally involve transforming plants with a DNA construct comprising a promoter that drives expression in a plant operably linked to at least a portion of a nucleotide sequence that is antisense to the transcript of the endogenous gene. Antisense nucleotides are constructed to hybridize with the corresponding mRNA. Modifications of the antisense sequences may be made as long as the sequences hybridize to and interfere with expression of the corresponding mRNA. In this manner, antisense constructions having at least about 75%, 80%, or 85% or more sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

Furthermore, catalytic RNA molecules or ribozymes can be used in combination with antisense suppression to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al. (1988) Nature 334:585-591.

In addition, a variety of cross-linking agents, alkylating agents, and radical generating species as pendant groups on polynucleotides of the present invention can be used to bind, label, detect, and/or cleave nucleic acids. For example, Vlassov et al. (1986) Nucleic Acids Res. 14:4065-4076, describe covalent bonding of a single-stranded DNA fragment with alkylating derivatives of nucleotides complementary to target sequences. A report of similar work by the same group is that by Knorre et al. (1985) Biochimie 67:785-789. Iverson and Dervan (1987) also showed sequence-specific cleavage of single-stranded DNA mediated by incorporation of a modified nucleotide which was capable of activating cleavage in (J. Am. Chem. Soc. 109:1241-1243). Meyer et al. (1989) J. Am. Chem. Soc. 111:8517-8519 effect covalent crosslinking to a target nucleotide using an alkylating agent complementary to the single-stranded target nucleotide sequence. A photoactivated crosslinking to single-stranded oligonucleotides mediated bypsoralen was disclosed by Lee et al. (1988) Biochem. 27:3197-3203. Use of crosslinking in triple-helix forming probes was also disclosed by Home et al. (1990) J. Am. Chem. Soc. 112:2435-2437. Use of N4, N4-ethanocytosine as an alkylating agent to crosslink to single-stranded oligonucleotides has also been described by Webb et al. (1986) J. Am. Chem. Soc. 108:2764-2765; Webb et al. (1986) Nucleic Acids Res. 14:7661-7674; Feteritz et al. (1991) J. Am. Chem. Soc. 113:4000. Various compounds to bind, detect, label, and/or cleave nucleic acids are known in the art. See, for example, U.S. Pat. Nos. 5,543,507; 5,672,593; 5,484,908; 5,256,648; and 5,681,941.

In another example of this embodiment of the invention, transgenic expression of an exogenous, functionally impaired MLH1 protein is used to inhibit the plant cellular mismatch repair system through competition with the endogenous plant MLH1 protein. This method comprises transforming a plant cell with at least one expression cassette comprising a promoter that drives expression in the plant operably linked to at least one nucleotide sequence encoding a functionally defective MLH 1 protein or variant thereof. “Functionally defective” is defined herein as having altered mismatch repair activity relative to the native plant MLH1 protein or a lack of mismatch repair activity. In this embodiment that the functionally defective MLH1 polypeptide may bind substrate with an affinity similar to that observed for the endogenous MLH1 enzyme, to allow for competition with the native enzyme or the functionally defective MLH1 polypeptide may interact with other components of the mismatch repair system and compete with wild-type MLH1 for the additional components required in mismatch repair including, but not limited to, MutS, MutH, Helicase II, MLH1 and their homologs. Similar to that described in previous embodiments, in this embodiment the inhibition of the cellular mismatch repair system is made transient, through the use of a chemical-inducible promoter to drive expression of the mutant MLH1 nucleotide sequence.

In another example of this embodiment of the invention, the previous three methods involving co-suppression, antisense suppression, and transgenic expression of an exogenous MLH1 protein are combined with elements of the FLP/FRT recombinase system. The FLP/FRT recombinase system is described in U.S. patent application Ser. No. 09/193,502, herein incorporated by reference. In this case, the FLP/FRT system is provided as an alternative to a chemical-inducible promoter to allow for the transient suppression of the plant cellular mismatch repair system.

This method comprises transforming a plant cell with a first expression cassette comprising a chemical-inducible promoter that drives expression in the plant cell operably linked to any of the sense or antisense MLH1 nucleotide sequences of the previously described three methods to inhibit the cellular mismatch repair system, wherein this first expression cassette is located between two FRT recombination sites or “FRT sequences” of the FLP/FRT recombinase system (U.S. patent application Ser. No. 09/193,502). The FRT sequences are oriented in such a manner as to allow for either inversion or excision of the expression cassette by FLP recombinase.

This method further comprises transformation of a plant with a second expression cassette, wherein a nucleotide sequence encoding FLP recombinase is operably linked to a second chemical-inducible promoter that drives expression in the plant. A plant comprising such a first and second expression cassette is then transformed with nucleic acid containing the desired mutation(s) or sequences to be homologously recombined in the presence of a chemical compound capable of inducing the promoter of the first expression cassette and, thus, inhibition of the cellular mismatch repair system. A chemical compound capable of inducing expression of FLP recombinase by the second chemical-inducible promoter is then added, resulting in FLP recombinase catalyzed excision or inversion of the first expression cassette and release of the inhibition of the cellular mismatch repair system. Transformed plants containing the mutated or recombined nucleic acid sequences are then selected as described supra.

In another example of this embodiment of the invention the plant cellular mismatch repair system is transiently suppressed and the efficiency of targeted gene mutation and homologous recombination increased through the use of an antibody that selectively binds to and inhibits the mismatch repair activity of an MLH1 protein. This method comprises transformation of the plant with nucleic acid containing the desired mutation(s) or sequences to be homologously recombined in the presence of an antibody that selectively binds to and inhibits the mismatch repair activity of an MHL1 protein and then selecting the transformed plants containing the mutated or recombined nucleic acid sequences.

Another embodiment of the present invention involves the production of hybrid plant species as described in U.S. Pat. No. 5,965,415, which is hereby incorporated in its entirety by reference. In this case, nucleic acid of a first species is transformed into a plant cell of a second species, wherein the cellular mismatch repair system of the second plant species has been impaired as described above in any one of the previous methods. In the manner detailed herein supra, the defective cellular mismatch repair activity allows for the non-homologous nucleic acid of the first species to be recombined with the chromosomic DNA of the second species creating a hybrid species. In the absence of functional mismatch repair activity, the E. coli chromosome can recombine with the chromosome of other bacteria such as S. tymphimurium or B. subtilis.

Another object of the present invention is the generation of plants that are reversibly male sterile as described in U.S. Pat. No. 6,072,102, which is hereby incorporated by reference in its entirety. In this case, male sterility is affected through the transient and tissue-specific inactivation of an MLH1 protein of the present invention. In this embodiment, the generation of reversible male sterility in a plant comprises the transformation of a plant with a first expression cassette consisting of a lexA DNA binding site embedded in a tissue-specific promoter that drives expression in the plant that is operatively linked to a sense or anti-sense nucleotide sequence corresponding to an MLH1 gene, wherein the nucleotide sequence when expressed disrupts pollen formation or function through inhibition of the cellular mismatch repair system. The method further comprises the transformation of a plant with a second expression cassette consisting of a nucleotide sequence encoding a lexA repressor protein operably linked to a chemical-inducible promoter that drives expression in a plant, wherein, when the plant is exposed to a compound capable of inducing the chemical-inducible promoter, the inhibition of the cellular mismatch repair system is released, and the male sterile effect is reversed.

In a related embodiment, the tissue specific promoter of the first expression cassette is an anther-specific promoter and the chemical-inducible promoter of the second expression cassette is a herbicidal safener as described in U.S. Pat. No. 6,072,102.

Another embodiment of the present invention is directed to methods for detecting and locating as little as a one base pair mismatch in a double-stranded nucleic acid molecule. Alterations in a nucleic acid sequence that are benign or have no negative consequences are sometimes called “polymorphisms”. In the present invention, alterations in the nucleic acid sequence, whether they have negative consequences or not, can be referred to as “mutations”. The methods of this invention have the capability to detect mutations regardless of biological effect or lack thereof. For the sake of simplicity, the term “mutation” is defined herein to mean an alteration in the base sequence of a nucleic acid strand compared to a reference strand. In the context of this invention, the term “mutation” includes the term “polymorphism” or any other similar or equivalent term of art. Furthermore, by a “base pair mismatch” is meant any base pairing in a double stranded nucleic acid molecule other than adenosine paired with thymidine or guanosine paired with cytidine. “Base pair mismatch” is used herein synonymously with “mismatch”, “mispair”, “base pair mutation”, and “polymorphism”. In addition, the phrase “double stranded nucleic acid molecule” is used herein interchangeably with the phrase “nucleic acid duplex”.

Methods involving the use of mismatch repair proteins for the detection, localization, and repair of base pair mismatches in double-stranded nucleic acid molecules have been described (see for example, U.S. Pat. No. 6,027,898 and U.S. Pat. No. 5,861,482, herein incorporated in their entirety by reference). In general, these methods involve contacting a double-stranded nucleic acid molecule with the components of the mismatch repair system, including but not limited to MutS, MutL, MutH, and Helicase II, and their homologs, such as the MutL Homolog 1 (MLH1) polypeptide of the invention, and then separating and detecting the specific nucleic acid:protein complex that is formed in the presence of a mutation. The methods further comprise a step in which the separated nucleic acid:protein complex is compared to a standard. MLH1 polypeptides of the present invention can be used with such methods for detecting mutations in double-stranded nucleic acids.

Generally, one example of a method for detecting and localizing a base pair mismatch in a nucleic acid duplex comprises contacting a double-stranded nucleic acid with a MLH1 polypeptide of the present invention under conditions such that the polypeptide forms specific complexes with MutS or homologs thereof, bound to mispaired bases in the nucleic acid duplex. The MLH1 polypeptide of the present invention may be used alone or in combination with other mismatch repair proteins including, but not limited to, MSH1 and PMS1. Any additional proteins or polypeptides that are present may be capable of cleaving the nucleic acid duplex at or near the site of the nucleic acid:protein complex. Alternatively, cleavage of the nucleic acid duplex in the vicinity of a mismatch can be enabled through modification of the MLH1 mismatch recognition polypeptide of the present invention by attachment of a hydroxyl radical cleavage function according to methods well known in the art and described in U.S. Pat. No. 5,459,039, herein incorporated by reference.

In a related example, the separation and detection of the nucleic acid:protein complex that is formed in the presence of a mutation involves the use of hydrolysis with an exonuclease. The nucleic acid molecules that have been contacted with the MLH1 polypeptide of the present invention under conditions such that the polypeptide forms specific complexes with polypeptides bound to mismatches are subjected to hydrolysis with an exonuclease under conditions such that the nucleic acid:protein complex blocks hydrolysis as described in U.S. Pat. No. 5,861,482. The location of the block to hydrolysis, the region of the mispair, is then determined by a suitable analytic method as described below.

The separation and detection of protein-complexed and uncomplexed nucleic acid molecules are performed according to various suitable analytical methods known in the art. For example, the separation and analysis of mixtures of nucleic acid molecules can be performed using techniques such as size exclusion chromatography, ion exchange chromatography, reverse phase chromatography, or Matched Ion Polynucleotide Chromatography. A change in the retention time or in the number of peaks in the chromatogram of the sample after contact with the mismatch repair polypeptide compared to the standard, indicates the presence of at least one mutation site. The standard is generally the nucleic acid sample prior to contact with the mismatch repair polypeptides. The change in retention time is a result of the binding of the mismatch repair proteins to the nucleic acid molecule, whereas a change in the number of peaks is a result of the cleavage of the nucleic acid duplex at or near the site of the mutation.

In a related example, separation of nucleic acid molecules containing at least one base pair mismatch from those that do not is enabled through the incorporation of biotin into the molecules that contain at least one mispair and then specifically removing them by binding to avidin. The labeling of the mismatch-containing nucleic acids is performed using a biotinylated nucleotide and a complete mismatch repair system as described in U.S. Pat. Nos. 6,027,898 and 5,861,482. Suitable analytical methods for determining the location of the nucleotide modification are known to those skilled in the art. Such a determination involves comparison of the modified nucleic acid molecules with homologous unmodified nucleic acid molecules.

Another method for detection of the nucleic acid:protein complex is the use an antibody specific for the MLH1 mismatch repair protein of the present invention. Antibodies specific for the MLH1 protein of the invention are prepared by standard immunological techniques known to those skilled in the art and described in Example 1. This method comprises separating the protein-complexed and uncomplexed nucleic acid molecules by immunoprecipitation with an antibody specific for an MLH1 polypeptide, and detecting the nucleic acid present in the precipitate.

Another aspect of this embodiment is the removal of nucleic acid molecules that contain one or more mismatches from a heterologous mixture of nucleic acid molecules, to obtain a homogeneous sample of mismatch-free nucleic acid molecules. In this embodiment either of the two previously described methods can be used. For example, nucleic acid molecules containing mutations are removed either through precipitation by binding to avidin, or through antibody immunoprecipitation. This embodiment has applications with, for example, PCR reactions.

The MLH1 sequences for use in the methods of the present invention are provided in expression cassettes for expression in the plant of interest. The cassette will include 5′ and 3′ regulatory sequences operably linked to a, for example, sense or anti-sense MLH1 sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the MLH1 sequence to be under the transcriptional regulation of the regulatory regions. The expression cassette may additionally contain selectable marker genes.

The expression cassette will include in the 5′-3′ direction of transcription, a transcriptional and translational initiation region, an MLH1 DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of the MLH1 mRNA in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the nucleotide sequence(s) of the invention may be optimized for increased expression in the transformed plant. That is, the nucleotide sequences of the invention can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff(1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

A number of promoters can be used in the practice of the invention. The promoters can be selected based on the desired outcome. The nucleic acids can be combined with constitutive, chemical-regulatable, or tissue-preferred, or other promoters for expression in plants, particularly a promoter that is chemical-inducible.

Such constitutive promoters include, for example, the core promoter of the Rsyn7 promoter and other constitutive promoters disclosed in WO 99/43838; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced protein expression within a particular plant tissue. Tissue-preferred promoters include Yamamoto et al. (1997) Plant J. 12(2)255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 promoter (WO 99/43838), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; 5,608,142; 6,177,611; herein incorporated by reference.

The present invention may be used for transformation of any plant species, including, but not limited to, monocots and dicots. However, the most preferable plants of the present invention are crop plants (for example, rice, corn, alfalfa, sunflower, Brassica, soybean, cotton, safflower, peanut, sorghum, wheat, millet, tobacco, etc.), particularly rice plants.

Examples of other plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

It is known that the compositions and methods of the present invention can be used to facilitate the genetic modification of plants to generate an unlimited range of plant phenotypes. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. application Ser. No. 08/838,763, filed Apr. 10, 1997; and U.S. Pat. Nos. 5,703,049, 5,885,801, and 5,885,802, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1 986) J. Biol. Chem. 261:6279; Kirihara et al. (1 98 8) Gene 71:3 59; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,93 1); avirulence (avr) and disease resistance (R) genes (Jones et al. (1 994) Science 266:789; Martin et al. (1 993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in copending U.S. application Ser. No. 08/838,763, filed Apr. 10, 1997, and U.S. Pat. Nos. 5,703,049, 5,885,801, and 5,885,802.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

Transformation protocols as well as protocols for introducing nucleotide sequences into plants may vary depending on the type of plant or plant cell, i.e., monocot or dicot, targeted for transformation. Protocols that are useful for transformation of rice plants in particular include biolistic methods (see Nayak et al. (1997) Proc. Natl. Acad. Sci. 94:2111-2116; and Christou, P. (1997) Plant Mol. Biol. 35:197-203) and Agrobacterium mediated methods (see Hiei et al. (1994) Plant J. 6:271-282; and Ishida et al. (1996) Nat. Biotechnol. 14:745-750). Additional suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. It is recognized that the protein of interest of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

In addition, the desired genetically altered trait can be bred into other plant lines possessing other desirable characteristics using conventional breeding methods and/or top-cross technology.

Methods for cross pollinating plants are well known to those skilled in the art, and are generally accomplished by allowing the pollen of one plant, the pollen donor, to pollinate a flower of a second plant, the pollen recipient, and then allowing the fertilized eggs in the pollinated flower to mature into seeds. Progeny containing the entire complement of heterologous coding sequences of the two parental plants can be selected from all of the progeny by standard methods available in the art as described infra for selecting transformed plants. If necessary, the selected progeny can be used as either the pollen donor or pollen recipient in a subsequent cross pollination.

### Experimental

### EXAMPLE 1

Preparation of Antibodies

Antibodies specific for MLH1 polypeptides of the present invention are produced by injecting female New Zealand white rabbits (Bethyl Laboratory, Montgomery, Tex.) six times with homogenized polyacrylamide gel slices containing 100 micrograms of PAGE purified MLH1 protein. Animals are then bled at two week intervals. The antibodies are further purified by affinity-chromatography with Affigel 15(BioRad)-immobilized antigen as described by Harlow et al. (1988) Antibodies: A Laboratory Manual, Cold Spring Harbor, N.Y.; incorporated herein in its entirety by reference. The affinity column is prepared with purified MLH1 protein essentially as recommended by BioRad.RTM. Immune detection of antigens on PVDF blots is carried out following the protocol of Meyer et al. (1988) J. Cell. Biol. 107:163; incorporated herein in its entirety by reference, using the ECL kit from Amersham (Arlington Heights, Ill.).

### EXAMPLE 2

Transformation of Rice Embryogenic Callus by Bombardment

Embryogenic callus cultures derived from the scutellum of germinating seeds serve as the source material for transformation experiments. This material is generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos is then transferred to CM media (N6 salts, Nitsch and Nitsch vitamins, 1 mg/l 2,4-D, Chu et al., 1985, Sci. Sinica 18:659-668). Callus cultures are maintained on CM by routine sub-culture at two week intervals and used for transformation within 10 weeks of initiation.

Callus is prepared for transformation by subculturing 0.5-1.0 mm pieces approximately 1 mm apart, arranged in a circular area of about 4 cm in diameter, in the center of a circle of Whatman #541 paper placed on CM media. The plates with callus are incubated in the dark at 27-28° C. for 3-5 days. Prior to bombardment, the filters with callus are transferred to CM supplemented with 0.25 M mannitol and 0.25 M sorbitol for 3 hr. in the dark. The petri dish lids are then left ajar for 20-45 minutes in a sterile hood to allow moisture on tissue to dissipate.

Circular plasmid DNA from two different plasmids one containing the selectable marker for rice transformation and one containing the nucleotide of the invention, are co-precipitated onto the surface of gold particles. To accomplish this, a total of 10 μg of DNA at a 2:1 ratio of trait:selectable marker DNAs is added to a 50 μl aliquot of gold particles resuspended at a concentration of 60 mg ml⁻¹. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are then washed twice with 1 ml of absolute ethanol and then resuspended in 50 μl of absolute ethanol and sonicated (bath sonicator) for one second to disperse the gold particles. The gold suspension is incubated at−70° C. for five minutes and sonicated (bath sonicator) if needed to disperse the particles. Six μl of the DNA-coated gold particles are then loaded onto mylar macrocarrier disks, and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen, and the callus is bombarded two times. Five to seven plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/l hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipet. Three ml aliquots of the callus suspension are plated onto fresh SM media and the plates incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% gelrite +50 ppm hyg B) for 2 weeks in the dark at 25° C. After 2 weeks the callus is transferred to RM2 media (MS salts, Nitsch and Nitsch vitamins, 3% sucrose, 0.4% gelrite+50 ppm hyg B) and placed under cool white light (˜40 μEm⁻²S⁻¹) with a 12 hr photoperiod at 25° C. and 30-40% humidity. After 2-4 weeks in the light, callus generally begins to organize, and form shoots. Shoots are removed from surrounding callus/media and gently transferred to RM3 media (1/2×MS salts, Nitsch and Nitsch vitamins, 1% sucrose +50 ppm hygromycin B) in phytatrays (Sigma Chemical Co., St. Louis, Mo.) and incubation is continued using the same conditions as described in the previous step.

Plants are transferred from RM3 to 4″ pots containing Metro mix 350 after 2-3 weeks, when sufficient root and shoot growth has occurred. Plants are grown using a 12 hr/12 hr light/dark cycle using ˜30/18° C. day/night temperature regimen.

### EXAMPLE 3

Transformation of Maize Embryos by Particle Bombardment

Immature maize embryos from greenhouse donor plants are bombarded with a plasmid containing a nucleotide sequence of the present invention operably linked to a selected promoter plus a plasmid containing the selectable marker gene PAT (Wohlleben et al. (1988) Gene 70:25-37) that confers resistance to the herbicide Bialaphos. Transformation is performed as follows.

Preparation of Target Tissue

The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 20 560Y medium for 4 hours and then aligned within the 2.5-cm target zone in preparation for bombardment.

Preparation of DNA

A plasmid vector comprising the nucleotide sequence of the present invention operably linked to a promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5” pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for the desired phenotypic trait.

### EXAMPLE 4

Agrobacterium-Mediated Transformation

For Agrobacterium-mediated transformation of maize, a nucleotide sequence of the present invention is operably linked to a selected promoter, and the method of Zhao is employed (U.S. Pat. No. 5,981,840, and International Publication No. WO 98/32326; the contents of which are hereby incorporated by reference). Briefly, immature embryos are isolated from maize and the embryos contacted with a suspension of Agrobacterium, where the bacteria are capable of transferring the nucleotide sequence of interest to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### EXAMPLE 5

Transformation of Soybean Embryos

Soybean embryos are bombarded with a plasmid containing a nucleotide sequence of the present invention operably linked to a selected promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures are maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the nucleotide sequence of the present invention operably linked to the selected promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media is exchanged with fresh media, and eleven to twelve days post-bombardment with fresh media containing 50 mg/ml hygromycin. This selective media is refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line is treated as an independent transformation event. These suspensions are then subcultured and maintained as clusters of immature embryos or regenerated into whole plants by maturation and germination of individual somatic embryos.

### EXAMPLE 6

Transformation of Sunflower Meristem Tissue

Sunflower meristem tissues are transformed with an expression cassette containing a nucleotide sequence of the present invention operably linked to a selected promoter as follows (see also European Patent Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Chlorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney et al. (1992) Plant Mol Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA1 05 is used in all transformation experiments. A binary plasmid vector comprising the expression cassette that contains the nucleotide sequence of the present invention operably linked to a selected promoter is introduced into Agrobacterium strain EHA105 via freeze-thawing as described by Holsters et al. (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of To plants (parental generation) maturing in the greenhouse are identified by NPTII ELISA analysis of leaf extracts while transgenic seeds harvested from NPTII-positive To plants are identified by the presence of the transgene of the invention in small portions of dry seed cotyledon.

An alternative sunflower transformation protocol allows the recovery of transgenic progeny without the use of chemical selection pressure. Seeds are dehulled and surface-sterilized for 20 minutes in a 20% Chlorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26 ° C. incubation conditions.

Explants (around 2 cm long) from two weeks of culture in 374C medium are screened for the presence of the transgene of the invention. After positive explants are identified, those shoots that fail to express the transgene of the invention are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate assay. At this time, the positive shoots recovered from a single node will generally have been enriched in the transgenic sector detected in the initial assay prior to nodal culture.

Recovered shoots positive for transgene expression are grafted to Pioneer hybrid 6440 in vitro-grown sunflower seedling rootstock. The rootstocks are prepared in the following manner. Seeds are dehulled and surface-sterilized for 20 minutes in a 20% Chlorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

### Bombardment and Culture Media

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂0 following adjustment to pH 5.8 with KOH); 2.0 g/l Gelrite (added after bringing to volume with D-I H₂0); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂0 following adjustment to pH 5.8 with KOH); 3.0 g/l Gelrite (added after bringing to volume with D-I H₂0); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂0) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂0 after adjusting to pH 5.6); 3.0 g/l Gelrite (added after bringing to volume with D-I H₂0); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂0), 0.1 g/l myo-inositol, and 40.0 g/l sucrose (brought to volume with polished D-I H₂0 after adjusting pH to 5.6); and 6 g/l bacto-agar (added after bringing to volume with polished D-I H₂0), sterilized and cooled to 60° C.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated nucleic acid molecule comprising a nucleotide sequence selected from the group consisting of:
(a) the nucleotide sequence shown in SEQ ID NO:1;
(b) a nucleotide sequence that encodes a polypeptide comprising the amino acid sequence of SEQ ID NO:2;
(c) a nucleotide sequence encoding an MLH1 polypeptide, wherein said nucleotide sequence hybridizes to the nucleotide sequence shown in SEQ ID NO:1 under stringent conditions;
(d) the cDNA insert of the plasmid deposited with ATCC as Patent Deposit No. PTA-2021;
(e) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(f) a nucleotide sequence encoding an MLH1 polypeptide, said sequence having at least about 75% sequence identity to the nucleotide sequence shown in SEQ ID NO:1;
(g) a nucleotide sequence encoding an MLH1 polypeptide having at least about 75% sequence identity to the polypeptide encoded by the cDNA insert of the plasmid deposited with ATCC as Patent Deposit No. PTA-2021;
(h) a nucleotide sequence encoding an MLH1 polypeptide having at least about 75% sequence identity to the polypeptide sequence shown in SEQ ID NO:2; and
(i) a nucleotide sequence comprising an antisense sequence corresponding to the nucleotide sequence in (a), (b), (c), (d), (e), (f), (g), or (h).

2. An expression cassette comprising a nucleic acid molecule of claim 1, wherein said nucleotide sequence is operably linked to a promoter that drives expression in a plant cell.

3. The expression cassette of claim 2, wherein said promoter is selected from the group consisting of constitutive, chemically regulatable, and tissue-preferred promoters.

4. An isolated nucleic acid molecule comprising a fragment of SEQ ID NO:1, said fragment comprising at least 27 contiguous nucleotides of a nucleotide sequence selected from the group consisting of:
(a) nucleotides 1-2283 of the nucleotide sequence of SEQ ID NO:1; and
(b) nucleotides 1-2283 of the cDNA insert of the plasmid deposited with ATCC as Patent Deposit No. PTA-2021.

5. An isolated nucleic acid molecule comprising a nucleotide sequence selected from the group consisting of:
(a) a nucleotide sequence comprising at least 60 nucleotides that encodes a fragment of the amino acid sequence set forth in SEQ ID NO:2; and
(b) a nucleotide sequence comprising at least 60 nucleotides that encodes a fragment of the amino acid sequence encoded by the cDNA insert of the plasmid deposited with ATCC as Patent Deposit No. PTA-2021.

6. A host cell engineered to express any one of the nucleic acid molecules of claim 1, 4, or 5.

7. An isolated polypeptide comprising an amino acid sequence selected from the group consisting of:
(a) the amino acid sequence set forth in SEQ ID NO:2;
(b) an amino acid sequence having at least 75% sequence identity to the amino acid sequence set forth in SEQ ID NO:2; and
(c) an amino acid sequence comprising at least 20 consecutive amino acids of the amino acid sequence set forth in (a) or (b).

8. A genetically modified rice plant comprising in its genome an endogenous MLH1 gene having a mutation within said gene, wherein said endogenous MLH1 gene corresponds to the cDNA set forth in SEQ ID NO:1 and said mutation is due to the presence of a transposon.

9. Genetically modified seed of said plant of claim 8.

10. A transformed plant comprising in its genome at least one stably incorporated expression cassette comprising a nucleotide sequence operably linked to a chemical-inducible promoter that drives expression in said plant cell, wherein said nucleotide sequence comprises a nucleotide sequence selected from the group consisting of:
(a) the nucleotide sequence shown in SEQ ID NO:1;
(b) a nucleotide sequence that encodes a polypeptide comprising the amino acid sequence of SEQ ID NO:2;
(c) a nucleotide sequence encoding an MLH1 polypeptide, wherein said nucleotide sequence hybridizes to the nucleotide sequence shown in SEQ ID NO:1 under stringent conditions;
(d) the cDNA insert of the plasmid deposited with ATCC as Patent Deposit No. PTA-2021;
(e) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(f) a nucleotide sequence encoding an MLH1 polypeptide, said sequence having at least about 75% sequence identity to the nucleotide sequence shown in SEQ ID NO:1;
(g) a nucleotide sequence encoding an MLH1 polypeptide having at least about 75% sequence identity to the polypeptide encoded by the cDNA insert of the plasmid deposited with ATCC as Patent Deposit No. PTA-2021;
(h) a nucleotide sequence encoding an MLH1 polypeptide having at least about 75% sequence identity to the polypeptide sequence shown in SEQ ID NO:2;
(i) a nucleotide sequence consisting of at least 27 consecutive nucleotides of nucleotides 1-2238 of (a) or (d); and
(j) a nucleotide sequence comprising an antisense sequence corresponding to the nucleotide sequence in (a), (b), (c), (d), (e), (f), (g), (h), or (i).

11. A transformed plant comprising in its genome:
(a) a first stably incorporated expression cassette comprising a nucleotide sequence operably linked to a promoter that drives expression in a plant cell, wherein said first expression cassette is located between two FRT sequences oriented to allow for inversion or excision of said first expression cassette by FLP recombinase, said nucleotide sequence selected from the group consisting of:
(i) the nucleotide sequence shown in SEQ ID NO:1;
(ii) a nucleotide sequence that encodes a polypeptide comprising the amino acid sequence of SEQ ID NO:2;
(iii) a nucleotide sequence encoding an MLH1 polypeptide, wherein said nucleotide sequence hybridizes to the nucleotide sequence shown in SEQ ID NO:1 under stringent conditions;
(iv) the cDNA insert of the plasmid deposited with ATCC as Patent Deposit No. PTA-2021;
(v) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(vi) a nucleotide sequence encoding an MLH1 polypeptide, said sequence having at least about 75% sequence identity to the nucleotide sequence shown in SEQ ID NO:1;
(vii) a nucleotide sequence encoding an MLH1 polypeptide having at least about 75% sequence identity to the polypeptide encoded by the cDNA insert of the plasmid deposited with ATCC as Patent Deposit No. PTA-2021;
(viii) a nucleotide sequence encoding an MLH1 polypeptide having at least about 75% sequence identity to the polypeptide sequence shown in SEQ ID NO:2;
(ix) a nucleotide sequence consisting of at least 27 consecutive nucleotides of nucleotides 1-2238 of (a) or (d); and
(x) a nucleotide sequence comprising an antisense sequence corresponding to the nucleotide sequence in (i), (ii), (iii), (iv), (v), (vi), (vii), (viii) or (ix); and
(b) a second stably incorporated expression cassette comprising a nucleotide sequence encoding said FLP recombinase operably linked to a chemical-inducible promoter that drives expression in said plant.

12. A transformed plant comprising in its genome at least one stably incorporated expression cassette, wherein said expression cassette comprises a nucleotide sequence operably linked to a heterologous chemical-inducible promoter that drives expression in said plant cell, wherein said nucleotide sequence encodes a mutated MLH1 polypeptide with defective mismatch repair activity due to mutagenesis of at least one amino acid residue necessary for normal mismatch repair activity, wherein said mutated MLH1 polypeptide binds substrate with an affinity similar to that observed for a corresponding non-mutated endogenous MLH1 enzyme.

13. A transformed plant comprising in its genome:
(a) a first stably incorporated expression cassette comprising a lexA DNA binding site embedded in a tissue-specific promoter that drives expression in a plant cell, wherein said tissue-specific promoter is operably linked to a first nucleotide sequence comprising a nucleotide sequence selected from the group consisting of:
(i) the nucleotide sequence shown in SEQ ID NO:1;
(ii) a nucleotide sequence that encodes a polypeptide comprising the amino acid sequence of SEQ ID NO:2;
(iii) a nucleotide sequence encoding an MLH1 polypeptide, wherein said nucleotide sequence hybridizes to the nucleotide sequence shown in SEQ ID NO:1 under stringent conditions;
(iv) the cDNA insert of the plasmid deposited with ATCC as Patent Deposit No. PTA-2021;
(v) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(vi) a nucleotide sequence encoding an MLH1 polypeptide, said sequence having at least about 75% sequence identity to the nucleotide sequence shown in SEQ ID NO:1;
(vii) a nucleotide sequence encoding an MLH1 polypeptide having at least about 75% sequence identity to the polypeptide encoded by the cDNA insert of the plasmid deposited with ATCC as Patent Deposit No. PTA-2021;
(viii) a nucleotide sequence encoding an MLH1 polypeptide having at least about 75% sequence identity to the polypeptide sequence shown in SEQ ID NO:2;
(ix) a nucleotide sequence consisting of at least 27 consecutive nucleotides of nucleotides 1-2238 of (a) or (d); and
(x) a nucleotide sequence comprising an antisense sequence corresponding to the nucleotide sequence in (i), (ii), (iii), (iv), (v), (vi), (vii), (viii) or (ix); and
(b) a second stably incorporated expression cassette comprising of a second nucleotide sequence encoding a lexA repressor operably linked to a chemical-inducible promoter that drives expression in a plant cell.

14. Transformed seed of the plant of any one of claim 10, 11, 12, or 13.

15. The transformed plant of any one of claims 10, 11, 12, or 13, wherein said plant is a monocot.

16. The transformed plant of claim 15, wherein said monocot is rice, maize, wheat, barley, sorghum, or rye.

17. A method for increasing the efficiency of targeted gene mutation or homologous recombination in a plant, said method comprising:
(a) transposon tagging an endogenous MLH1 gene in said plant;
(b) transforming said plant with nucleic acid comprising a nucleotide sequence having at least one desired mutation or at least one nucleotide sequence to be homologously recombined; and
(c) selecting said transformed plants that contain said mutation or said homologously recombined nucleotide sequence.

18. The method of claim 17, wherein said plant is rice and wherein said MLH1 gene corresponds to the nucleotide sequence set forth in SEQ ID NO:1.

19. A method for increasing the efficiency of targeted gene mutation or homologous recombination in a plant, said method comprising:
(a) transforming said plant with at least one expression cassette comprising a nucleotide sequence operably linked to a chemical-inducible promoter that drives expression in a plant cell, wherein said nucleotide sequence comprises a nucleotide sequence selected from the group consisting of:
(i) the nucleotide sequence shown in SEQ ID NO:1;
(ii) a nucleotide sequence that encodes a polypeptide comprising the amino acid sequence of SEQ ID NO:2;
(iii) a nucleotide sequence encoding an MLH1 polypeptide, wherein said nucleotide sequence hybridizes to the nucleotide sequence shown in SEQ ID NO:1 under stringent conditions;
(iv) the cDNA insert of the plasmid deposited with ATCC as Patent Deposit No. PTA-2021;
(v) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(vi) a nucleotide sequence encoding an MLH1 polypeptide, said sequence having at least about 75% sequence identity to the nucleotide sequence shown in SEQ ID NO:1;
(vii) a nucleotide sequence encoding an MLH1 polypeptide having at least about 75% sequence identity to the polypeptide encoded by the cDNA insert of the plasmid deposited with ATCC as Patent Deposit No. PTA-2021;
(viii) a nucleotide sequence encoding an MLH1 polypeptide having at least about 75% sequence identity to the polypeptide sequence shown in SEQ ID NO:2;
(ix) a nucleotide sequence consisting of at least 27 consecutive nucleotides of nucleotides 1-2238 of (a) or (d); and
(x) a nucleotide sequence comprising an antisense sequence corresponding to the nucleotide sequence in (i), (ii), (iii), (iv), (v), (vi), (vii), (viii) or (ix);
(b) transforming said plant with nucleic acid comprising a nucleotide sequence having at least one desired mutation or at least one nucleotide sequence to be homologously recombined, wherein said transforming occurs in the presence of a chemical compound capable of inducing said chemical-inducible promoter, whereby said plant's cellular mismatch repair system is inhibited; and
(c) selecting said transformed plants that contain said mutation or said homologously recombined nucleotide sequence.

20. A method for increasing the efficiency of targeted gene mutation or homologous recombination in a plant, said method comprising:
(a) transforming said plant with a first expression cassette comprising a nucleotide sequence operably linked to a first chemical-inducible promoter that drives expression in a plant cell, wherein said first expression cassette is located between two FRT sequences oriented to allow for inversion or excision of said first expression cassette by FLP recombinase; wherein said nucleotide sequence is selected from the group consisting of:
(i) the nucleotide sequence shown in SEQ ID NO:1;
(ii) a nucleotide sequence that encodes a polypeptide comprising the amino acid sequence of SEQ ID NO:2;
(iii) a nucleotide sequence encoding an MLH1 polypeptide, wherein said nucleotide sequence hybridizes to the nucleotide sequence shown in SEQ ID NO:1 under stringent conditions;
(iv) the cDNA insert of the plasmid deposited with ATCC as Patent Deposit No. PTA-2021;
(v) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(vi) a nucleotide sequence encoding an MLH1 polypeptide, said sequence having at least about 75% sequence identity to the nucleotide sequence shown in SEQ ID NO:1;
(vii) a nucleotide sequence encoding an MLH1 polypeptide having at least about 75% sequence identity to the polypeptide encoded by the cDNA insert of the plasmid deposited with ATCC as Patent Deposit No. PTA-2021;
(viii) a nucleotide sequence encoding an MLH1 polypeptide having at least about 75% sequence identity to the polypeptide sequence shown in SEQ ID NO:2;
(ix) a nucleotide sequence consisting of at least 27 consecutive nucleotides of nucleotides 1-2238 of (a) or (d); and
(x) a nucleotide sequence comprising an antisense sequence corresponding to the nucleotide sequence in (i), (ii), (iii), (iv), (v), (vi), (vii), (viii) or (ix);
(b) transforming said plant with a second expression cassette comprising a nucleotide sequence encoding said FLP recombinase operably linked to a second chemical-inducible promoter that drives expression in said plant;
(c) transforming said plant with nucleic acid comprising a nucleotide sequence having at least one desired mutation or at least one nucleotide sequence to be homologously recombined in the presence of a chemical compound capable of inducing expression by said first chemical-inducible promoter, whereby said plant's cellular mismatch repair system is inhibited;
(d) contacting said plant with a chemical compound capable of inducing expression of said second chemical-inducible promoter thereby inducing expression of FLP recombinase to release said inhibition of the cellular mismatch repair system; and
(e) selecting said transformed plants containing said mutation or said homologously recombined nucleotide sequence.

21. A method for increasing the efficiency of targeted gene mutation or homologous recombination in a plant, said method comprising:
(a) transforming said plant with nucleic acid comprising a nucleotide sequence having at least one desired mutation or at least one sequence to be homologously recombined in the presence of an antibody that selectively binds to and inhibits mismatch repair activity of a polypeptide comprising an amino acid sequence selected from the group consisting of:
(i) the amino acid sequence set forth in SEQ ID NO:2;
(ii) an amino acid sequence comprising at least 75% sequence identity to the amino acid sequence set forth in SEQ ID NO:2; and
(iii) an amino acid sequence comprising at least 20 consecutive amino acids of the amino acid sequence set forth in (i) or (ii); and
(b) selecting said plants containing said mutation or said homologously recombined nucleotide sequence.

22. A method for increasing the efficiency of targeted gene mutation or homologous recombination in a plant, said method comprising:
(a) transforming said plant with at least one expression cassette comprising a nucleotide sequence operably linked to a heterologous chemical-inducible promoter that drives expression in said plant cell, wherein said nucleotide sequence encodes a mutated MLH1 polypeptide with defective mismatch repair activity due to mutagenesis of at least one amino acid residue necessary for normal mismatch repair activity, wherein said mutated MLH1 polypeptide binds substrate with an affinity similar to that observed for a corresponding non-mutated endogenous MLH1 enzyme;
(b) transforming said plant with nucleic acid comprising a nucleotide sequence having at least one desired mutation or at least one sequence to be homologously recombined, wherein said transforming occurs in the presence of a chemical compound capable of inducing said chemical-inducible promoter, thereby inducing expression of said MLH1 polypeptide with defective mismatch repair activity; and
(c) selecting said plants that contain said mutation or said homologously recombined nucleotide sequence.

23. The method of any one of claim 17, 19, 20, 21, or 22, wherein said nucleic acid comprising the nucleotide sequence having the desired mutation or the nucleotide sequence to be homologously recombined is that of a species different from said plant being transformed, whereby a hybrid plant species is formed.

24. A method for detecting, locating, or removing at least one base pair mismatch in a double-stranded nucleic acid molecule, said method comprising:
(a) providing a nucleic acid duplex comprising at least one base pair mismatch;
(b) contacting said nucleic acid duplex with an isolated polypeptide possessing MLH1 mismatch recognition activity, either alone or in combination with other mismatch repair proteins, wherein said polypeptide comprises an amino acid sequence selected from the group consisting of:
(i) the amino acid sequence set forth in SEQ ID NO:2;
(ii) an amino acid sequence having at least 75% sequence identity to the amino acid sequence set forth in SEQ ID NO:2; and
(iii) an amino acid sequence comprising at least 20 consecutive amino acids of the amino acid sequence set forth in (i) or (ii); and
(c) detecting any complex between said nucleic acid duplex and said polypeptide as a measure of the presence of said base pair mismatch in the nucleic acid duplex.

25. The method of claim 24, wherein the detection or removal of said complex comprises the use of an antibody that binds selectively to said polypeptide.

26. The method of claim 24, wherein said base pair mismatch is a SNP.

27. A method for producing reversible male sterility in a plant, said method comprising:
(a) transforming a plant with a first expression cassette comprising of a lexA DNA binding site embedded in a tissue-specific promoter that drives expression in said plant operably linked to a first nucleotide sequence that when expressed disrupts pollen formation or function through inhibition of said plant's cellular mismatch repair system, wherein said first nucleotide sequence is selected from the group consisting of:
(i) the nucleotide sequence shown in SEQ ID NO:1;
(ii) a nucleotide sequence that encodes a polypeptide comprising the amino acid sequence of SEQ ID NO:2;
(iii) a nucleotide sequence encoding an MLHl polypeptide, wherein said nucleotide sequence hybridizes to the nucleotide sequence shown in SEQ ID NO:1 under stringent conditions;
(iv) the CDNA insert of the plasmid deposited with ATCC as Patent Deposit No. PTA-2021;
(v) a nucleotide sequence encoding an MLH1 polypeptide, wherein said nucleotide sequence hybridizes to the cDNA insert of the plasmid deposited with ATCC as Patent Deposit No. PTA-2021 under stringent conditions;
(vi) a nucleotide sequence encoding an MLH1 polypeptide, said sequence having at least about 75% sequence identity to the nucleotide sequence shown in SEQ ID NO:1;
(vii) a nucleotide sequence encoding an MLH1 polypeptide having at least about 75% sequence identity to the polypeptide encoded by the cDNA insert of the plasmid deposited with ATCC as Patent Deposit No. PTA-2021;
(viii) a nucleotide sequence encoding an MLH1 polypeptide having at least about 75% sequence identity to the polypeptide sequence shown in SEQ ID NO:2;
(ix) a nucleotide sequence consisting of at least 27 consecutive nucleotides of nucleotides 1-2238 of (a) or (d); and
(x) a nucleotide sequence comprising an antisense sequence corresponding to the nucleotide sequence in (i), (ii), (iii), (iv), (v), (vi), (vii), (viii) or (ix);
(c) transforming said plant with a second expression cassette comprising a second nucleotide sequence encoding a lexA repressor protein operably linked to a chemical-inducible promoter that drives expression in said plant; and
(d) exposing said plant to a compound capable of inducing said chemical-inducible promoter, thereby inducing expression of said lexA repressor protein, whereby inhibition of the cellular mismatch repair system is released and said male sterility is reversed.

28. The method of claim 27, wherein said tissue-specific promoter is an anther-specific promoter and said chemical-inducible promoter is a herbicidal safener.

29. An antibody that binds selectively to a polypeptide selected from the group consisting of:
(a) a polypeptide comprising the amino acid sequence set forth in SEQ ID NO:2;
(b) a polypeptide comprising at least 75% sequence identity to the amino acid sequence set forth in SEQ ID NO:2; and
(c) a polypeptide comprising at least 20 consecutive amino acids of the amino acid sequence set forth in (a) or (b).
